(19) United States
(12) Patent Application Publication
(10) Pub. No.: US 2003/0017566 A1
(43) Pub. Date: Jan. 23, 2003

(54) Maize peroxidase genes and their use for improving plant disease resistance and stalk strength

(75) Inventors: Jon P. Duvick, Des Moines, IA (US); Joyce R. Maddox, Omaha, NE (US); Pedro A. Navarro Acevedo, Ames, IA (US); Carl R. Simmons, Des Moines, IA (US)

Correspondence Address: ALSTON & BIRD LLP, PIONEER HI-BRED INTERNATIONAL, INC., BANK OF AMERICA PLAZA, 101 SOUTH TYRON STREET, SUITE 4000, CHARLOTTE, NC, 28280-4000, US

(73) Assignee: Pioneer Hi-Bred International, Inc.

(21) Appl. No.: 10/047,825

(22) Filed: Jan. 16, 2002

Related U.S. Application Data
(60) Provisional application No. 60/262,595, filed on Jan. 18, 2001.

(51) Int. Cl. C12N 9/02; C07H 21/04; C12P 21/02; C12N 5/06

(52) U.S. Cl. 435/189; 435/320.1; 435/325; 435/69.1; 536/23.2

(57) ABSTRACT

Methods and compositions for modulating the plant defense response are provided. Nucleotide and amino acid sequences for maize peroxidases are provided. These sequences can be used in expression cassettes for modulating plant defense response, increasing plant disease resistance and increasing plant stalk strength. These sequences can also be used in methods of selecting or breeding for plants with increased disease resistance. Transformed plants, plant cells, tissues, and seed are also provided.

### CROSS-REFERENCE TO RELATED APPLICATIONS

This application claims the benefit of U.S. Provisional Application No. 60/262,595, filed Jan. 18, 2001.

### FIELD OF THE INVENTION

The invention relates to the field of the genetic manipulation of plants, particularly to the enhancement of disease resistance and stalk strength in plants.

### BACKGROUND OF THE INVENTION

Disease in plants is caused by biotic and abiotic causes. Biotic causes include fungi, viruses, bacteria, and nematodes. An example of the importance of plant disease is illustrated by phytopathogenic fungi, which cause significant annual crop yield losses. Plant disease outbreaks have resulted in catastrophic crop failures that have triggered famines and caused major social change. All of the approximately 300,000 species of flowering plants are attacked by pathogenic fungi; however, a single plant species can be host to only a few fungal species, and similarly, most fungi usually have a limited host range. Generally, the best strategy for plant disease control is to use resistant cultivars selected or developed by plant breeders for this purpose. However, the potential for serious crop disease epidemics persists today, as evidenced by recent outbreaks of the Victoria blight of oats and southern corn leaf blight. Genetic engineering of crops allows the implementation of novel mechanisms for disease resistance and allows resistance to be introduced more quickly than traditional breeding methods. Accordingly, molecular methods are needed to supplement traditional breeding methods to produce plants resistant to pathogen attack.

A host of cellular processes enable plants to defend themselves against disease caused by pathogenic agents. These defense mechanisms are activated by initial pathogen infection in a process known as elicitation. In elicitation, the host plant recognizes a pathogen-derived compound known as an elicitor; the plant then activates disease gene expression to limit further spread of the invading microorganism. It is generally believed that to overcome these plant defense mechanisms, plant pathogens must find a way to suppress elicitation as well as to overcome more physically-based barriers to infection, such as reinforcement and/or rearrangement of the actin filament networks near the cell's plasma membrane.

The present invention addresses the need for methods of increasing plant disease resistance by identifying novel nucleotide sequences and polypeptides that can be used to enhance a plant's defensive elicitation response.

### SUMMARY OF THE INVENTION

The present invention encompasses compositions and methods useful for enhancing plant disease resistance. Particularly, the nucleotide and amino acid sequences for eighteen maize peroxidase coding sequence are provided. Host cells, plants, plant tissues and seed transformed with the peroxidase-encoding nucleotide sequences are also provided. In some embodiments, the transformed plants and seed are monocotyledonous, while in other embodiments the transformed plants and seed are dicotyledonous.

The present invention also provides methods for modulating (i.e. increasing or decreasing) the defense response in a plant. The methods comprise stably transforming a plant with at least one peroxidase nucleotide sequence of the invention that is operably linked with a promoter capable of driving expression of the nucleotide sequence in a plant cell. In one embodiment, the promoter is a constitutive promoter, while in another embodiment, the promoter is a pathogen-inducible promoter.

The peroxidase-encoding nucleotide sequences of the invention may also be used in a method of selecting for or breeding for plants with increased disease resistance.

The peroxidase-encoding nucleotide sequences of the invention may also be used in methods of increasing the stalk strength of a plant.

The peroxidase-encoding nucleotide sequences of the invention may also be used in methods of preventing oxidative damage following anoxia in a plant.

### BRIEF DESCRIPTION OF THE DRAWINGS

FIG. 1A-D shows a CLUSTAL W alignment of the amino acid sequences of the novel peroxidase molecules of the invention. The amino acid sequences shown in the figure are set forth in SEQ ID NOS:2, 4, 6,8, 10, 12, 14, 17, 19, 21, 23, 25, 27, 29, 31, 33, 35, and 37.

### DETAILED DESCRIPTION OF THE INVENTION

The present invention provides, inter alia, compositions and methods for modulating the total level of proteins of the present invention and/or altering their ratios in a plant. By “modulation” is intended an increase or decrease in a particular character, quality, substance, or response.

The compositions of the invention comprise maize peroxidase nucleotide sequences and polypeptides. Peroxidases are a subclass of oxido-reductases that use a peroxide such as H₂O₂ as an oxygen acceptor. In plants, peroxidases are monomeric proteins whose activities are closely regulated by the plant. Peroxidases function in the synthesis of plant cell walls by promoting the polymerization of the monolignols coniferyl, p-coumaryl, and sinapyl alcohol into lignin. Lignification serves to strengthen and reinforce plant cell walls, and increase the stalk strength of the plant. Plant peroxidases are also required for xenobiotic detoxification (reviewed in Korte et al. (2000) Ecotoxicol. Environ. Saf. 47:1-26).

Although the present invention is not intended to be limited by its mechanism of action, peroxidases function in the plant defense response in various ways. For example, a plant undergoing an attack by a pathogen often produces a burst of reactive oxygen species (ROS) including peroxides. This ROS burst is believed to be an adaptive mechanism for combating the pathogen. The burst of ROS creates stress in the plant tissues, and peroxidases function to mitigate or control the ROS burst such that antipathogenic activity is maximized while toxicity to the plant is minimized.

Peroxidases prevent oxidative damage following anoxia (i.e. oxygen deprivation) in plants (Amor et al. (2000) FEBS Lett. 477:175-180). Anoxia followed by reoxygenation causes extensive damage to cellular components through the generation of reactive oxygen intermediates. However, anoxia pretreatment protected soybean (but not fibroblasts) again peroxide concentrations that induced programmed cell death in normoxic cells. This protection involved an increase in the expression of alternative oxidase (AOX) and peroxidases. Ascorabate peroxidases have also been shown to play a role in protecting against oxidative stress (Wang et al. (1999) Plant Cell Physiol. 40:725-32). The expression of the peroxisomal ascorbate peroxidase APX3 was demonstrated to protect tobacco leaves from oxidative stress damage caused by aminotriazole.

Peroxidases generate secondary metabolites that may have antipathogenic activity and contribute to a plant's defense mechanism. Additionally, peroxidases' role in lignin formation improves cell wall strength, hence increasing resistance to pathogen attack.

Several lines of experimental evidence support the role of peroxidases in the plant defense response. Induction of peroxidase activity is seen in Vigna sinensis L., Lycopersicon esculentum, and Stylosanthes humilis after exposure to fungal pathogens (Fink et al. (1991) Planta 185:246-254, Anfoka and Buchenauer (1997) Physiol. Mol. Plant. Pathol. 50:85-101, and Curtis et al. (1997) Mol. Plant Microb. Interact. 10:326-338); in Medicago truncatula following infection by Rhizhobium (Cook et al. (1995) Plant Cell 7:43-55); in tobacco following wounding (Hiraga-Susumu et al. (2000) Plant Cell Physiol. 41:165-170); and in Lycopersicon esculentum following injury by third-instar Helicoverpa zea larva (Stout et aL (1999) Physiol. Mol. Plant Pathol. 54:115-130).

Plant cells undergoing senescence shows changes in the level of peroxidase expression. For example, root nodules that are undergoing premature- senescence induced by exposure to high levels of salinity show an accompanying decrease in the expression of peroxide scavenging enzymes including catalase, and ascorbate peroxidase (Swaraj and Bishnoi (1999) Indian J. Exp. Biol. 37:843-848). In fact, the expression or activity of plant peroxidases has been used in the art as a marker for senescence. See, for example, Oh et al. (1997) Plant J. 12:527-535, Clendennen and May (1997) Plant Physiol. 115:463-469, Toumaire et al. (1996) Plant Physiol 111:159-168, and Gorin and Heidema (1976) J. Agric. Food Chem. 24:200-201; herein incorporated by reference.

The nucleotide and amino acid sequences of eighteen novel peroxidases from Zea mays are disclosed in the present invention. The peroxidase nucleotide sequences include Zm-POX01 (SEQ ID NO:1), Zm-POX04 (SEQ ID NO:3), ZmPOX05 (SEQ ID NO:5), Zm-POX06 (SEQ ID NO:7) Zm-POX07 (SEQ ID NO:9), Zm-POX08 (SEQ ID NO:1), Zm-POX10 (SEQ ID NO:13), Zm-POX16 (SEQ ID NO:16), Zm-POX17 (SEQ ID NO:18), Zm-POX18 (SEQ ID NO:20), Zm-POX20 (SEQ ID NO:22), Zm-POX21 (SEQ ID NO:24), Zm-POX24 (SEQ ID NO:26), Zm-POX26 (SEQ ID NO:28), Zm-POX28 (SEQ ID NO:30), Zm-POX31 (SEQ ID NO:32), Zm-POX34 (SEQ ID NO:34), and Zm-POX37 (SEQ ID NO:36). The peroxidase amino acid sequences encoded by these nucleotide sequences are set forth in SEQ ID NOS: 2 (Zm-POX01), 4(Zm-POX04), 6(Zm-POX05), 8(Zm-POX06), 10(Zm-POX07), 12(Zm-POX08), 14(Zm-POX10), 17(Zm-POX16), 19(Zm-POX17), 21(Zm-POX18), 23(Zm-POX20), 25(Zm-POX21), 27(Zm-POX24), 29(Zm-POX26), 31(Zm-POX28), 33(Zm-POX31), 35(Zm-POX34), and 37(Zm-POX37), respectively. The Zm-POX16 nucleotide sequence is also found in a form containing an unspliced intron (SEQ ID NO:15)

Peroxidase are generally classified as either basic, acidic, or neutral, based on their pI's. Twelve of the peroxidase polypeptides of the present invention (Zm-POX37, Zm-POX20, Zm-POX05, Zm-POX10, Zm-POX21, Zm-POX16, Zm-POX01, Zm-POX08, Zm-POX26, Zm-POX24, Zm-POX17, and Zm-POX34) are acidic (anionic), while six (Zm-POX18, Zm-POX07, Zm-POX04, Zm-POX28, Zm-POX06, and Zm-POX31) are basic (cationic). Induction of cationic peroxidases has been observed in incompatible resistance interactions between rice and Xanthomonas oryzae pv oryzae (Reimers et al. (1992) Plant Physiol. 99:1044-1050).

The peroxidase sequences of the present invention may be used to enhance the plant pathogen defense system. Plant peroxidase genes modulate the effects of oxidative burst that comprises part of the early defense response in plants. Plant peroxidases also function in strengthening the plant cell wall by promoting lignin formation. Hence, the compositions and methods of the invention can be used for enhancing resistance to plant pathogens including fungal pathogens, plant viruses, and the like. The method involves stably transforming a plant with a nucleotide sequence capable of modulating the plant pathogen defense system operably linked with a promoter capable of driving expression of a gene in a plant cell.

Compositions

Compositions of the invention include the sequences for eighteen maize nucleotide sequences which have been identified as members of the peroxidase family in maize that are involved in defense response and cell wall strength.

The present invention provides for isolated nucleic acid molecules comprising nucleotide sequences encoding the amino acid sequences shown in SEQ ID NOS: 2, 4, 6, 8, 10, 12, 14, 17, 19, 21, 23, 25, 27, 29, 31, 33, 35, or 37. Further provided are polypeptides having an amino acid sequence encoded by a nucleic acid molecule described herein, for example the nucleotide sequences set forth in SEQ ID NO: 1, 3, 5, 7, 9, 11, 13, 15, 16, 18, 20, 22, 24, 26, 28, 30, 32, 34, and 36, and fragments and variants thereof.

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence have peroxidase-like activity and thereby affect development, developmental pathways, and defense responses. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, about 200 nucleotides, and up to the full-length nucleotide sequence encoding the proteins of the invention.

A fragment of peroxidase nucleotide sequence that encodes a biologically active portion of a peroxidase polypeptide of the invention will encode at least 15, 25, 30, 50, 100, 150, 200, 250, or 300 contiguous amino acids, or up to the total number of amino acids present in a full-length peroxidase polypeptide of the invention (for example, 219 amino acids for SEQ ID NO:2, 313 amino acids for SEQ ID NO:4, 356 amino acids for SEQ ID NO:6, 358 amino acids for SEQ ID NO:8, 346 amino acid for SEQ ID NO:10, 339 amino acids for SEQ ID NO:12, 347 amino acids for SEQ ID NO:14, 362 amino acids for SEQ ID NO:17, 328 amino acids for SEQ ID NO:19, 327 amino acids for SEQ ID NO:21, 342 amino acids for SEQ ID NO:23, 337 amino acids for SEQ ID NO:25, 320 amino acids for SEQ ID NO:27 and SEQ ID NO:29, 332 amino acids for SEQ ID NO:31, 355 amino acids for SEQ ID NO:33, 328 amino acids for SEQ ID NO:35, and 325 amino acids for SEQ ID NO:37). Fragments of a peroxidase nucleotide sequence that are useful as, for example, hybridization probes or polymerase chain reaction (PCR) primers generally need not encode a biologically active portion of a peroxidase polypeptide.

Thus, a fragment of a peroxidase nucleotide sequence may encode a biologically active portion of a peroxidase polypeptide, or it may be a fragment that can be used as a hybridization probe or PCR primer using methods disclosed herein. A biologically active portion of a peroxidase protein can be prepared by isolating a portion of one of the peroxidase nucleotide sequences of the invention, expressing the encoded portion of the peroxidase protein (e.g., by recombinant expression in vitro), and assessing the activity of the encoded portion of the peroxidase protein. Nucleic acid molecules that are fragments of a peroxidase nucleotide sequence comprise at least 16, 20, 50, 75, 100, 150, 200, 250, 300, 350, 400, 450, 500, 550, 600, 650, 700, 800, 900, 1000, 1100, 1200, 1300, or 1400 nucleotides, or up to the number of nucleotides present in a full-length peroxidase nucleotide sequence disclosed herein (for example, 831 nucleotides for SEQ ID NO:1, 1354 nucleotides for SEQ ID NO:3, 1263 nucleotides for SEQ ID NO:5, 1519 nucleotides for SEQ ID NO:7, 1480 nucleotides for SEQ ID NO:9, 1183 nucleotides for SEQ ID NO:11, 1407 nucleotides for SEQ ID NO:13, 1565 nucleotides for SEQ ID NO:15, 1388 nucleotides for SEQ ID NO:16, 1467 nucleotides for SEQ ID NO:18, 1522 nucleotides for SEQ ID NO:20, 1451 nucleotides for SEQ ID NO:22, 1334 nucleotides for SEQ ID NO:24, 1285 nucleotides for SEQ ID NO:26, 1159 nucleotides for SEQ ID NO:28, 1310 nucleotides for SEQ ID NO:30, 1170 nucleotides for SEQ ID NO:32, 1391 nucleotides for SEQ ID NO:34, and 1476 nucleotides for SEQ ID NO:36).

By “variants” is intended substantially similar sequences. For nucleotide sequences, conservative variants include those sequences that, because of the degeneracy of the genetic code, encode the amino acid sequence of one of the peroxidase polypeptides of the invention. Naturally occurring allelic variants such as these can be identified with the use of well-known molecular biology techniques, as, for example, with PCR and hybridization techniques as outlined herein. Variant nucleotide sequences also include synthetically-derived nucleotide sequences, such as those generated, for example, by using site-directed mutagenesis but which still encode a peroxidase polypeptide of the invention. Generally, variants of a particular nucleotide sequence of the invention will have at least about 40%, 50%, 60%, 65%, 70%, generally at least about 75%, 80%, 85%, preferably at least about 90%, 91%, 92%, 93%, 94%, 95%, 96%, 97and more preferably at least about 98%, 99% or more sequence identity to that particular nucleotide sequence as determined by sequence alignment programs described elsewhere herein using default parameters.

By “variant” protein or polypeptide is intended a protein or polypeptide derived from the native protein or polypeptide by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, peroxidase-like activity as described herein. Such variants may result from, for example, genetic polymorphism or from human manipulation. Biologically active variants of a native peroxidase protein of the invention will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

Biological activity of the peroxidase polypeptides (i.e., influencing the plant defense response or stalk strength) can be assayed by any method known in the art. Peroxidase-like activity may be assayed, for example, as described by Lagrimini and Rothstein (1987) Plant Physiol. 84:438-442, herein incorporated by reference. Stalk strength may also be measured, for example, by the method described in U.S. Pat. No. 5,044,210, herein incorporated by reference.

The polypeptides of the invention may be altered in various ways including by amino acid substitutions, deletions, truncations, and insertions. Novel polypeptides having properties of interest may be created by combining elements and fragments of polypeptides of the present invention as well as other polypeptides. Methods for such manipulations are generally known in the art. For example, amino acid sequence variants of the peroxidase polypeptides can be prepared by mutagenesis of the nucleotide sequences that encodes these polypeptides. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the peroxidase polypeptides may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

Also encompassed are peroxidase variants in which key residues or domains have been mutated or shuffled (e.g. exchanged between related sequences) such that substrate specificity is altered or catalytic activity is enhanced. Methods of modifying peroxidase polypeptides to alter substrate specificity and stability are known in the art. See, for example, Mareeva et al. (1996) Appl. Biochem. Biotechnol. 61:13-23; herein incorporated by reference.

Thus, the nucleotide sequences of the invention include both the naturally occurring sequences as well as mutant forms. Likewise, the polypeptides of the invention encompass both naturally occurring polypeptides as well as variations and modified forms thereof. Such variants will continue to possess the desired defense response activity or stalk-strengthening activity. The mutations to be made in the nucleotide sequence encoding the variant must not place the sequence out of reading frame and preferably will not create complementary regions that could produce secondary mRNA structure. See, for example, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by peroxidase activity assays or stalk strength assays as described elsewhere herein. Additionally, differences in the expression of specific genes between uninfected and infected plants can be determined using gene expression profiling. RNA was analyzed using the gene expression profiling process (GeneCalling®) as described in U.S. Pat. No. 5,871,697, herein incorporated by reference.

Variant nucleotide sequences and proteins also encompass sequences and proteins derived from a mutagenic and recombinogenic procedure such as DNA shuffling. With such a procedure, one or more different peroxidase coding sequences can be manipulated to create a new peroxidase polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the peroxidase gene of the invention and other known peroxidase genes to obtain a new gene coding for a protein with an improved property of interest, such as an increased Km in the case of an enzyme. Such shuffling of domains may also be used to assemble novel proteins having novel properties. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire peroxidase sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as p, or any other detectable marker. Thus, for example, probes for hybridization can be made by labeling synthetic oligonucleotides based on the peroxidase sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire peroxidase nucleotide sequence disclosed herein, or one or more portions thereof, may be used as a probe capable of specifically hybridizing to corresponding peroxidase sequences and messenger RNAs. To achieve specific hybridization under a variety of conditions, such probes include sequences that are unique among peroxidase sequences and are preferably at least about 10 nucleotides in length, and most preferably at least about 20 nucleotides in length. Such probes may be used to amplify corresponding sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12 hours. Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tm can be approximated from the equation of Meinkoth and Wahl (1984) Anal. Biochem. 138:267-284: Tₘ=81.5° C.+16.6 (log M)+0.41 (%GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated nucleotide sequences that encode a peroxidase polypeptide and which hybridize under stringent conditions to the peroxidase sequences disclosed herein, or to fragments thereof, are encompassed by the present invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1 990) supra. BLAST nucleotide searches can be performed with the BLASTN program, score=100, wordlength 12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.hlm.nih.gov. Alignment may also be performed manually by inspection.

Unless otherwise stated, sequence identity/similarity values provided herein refer to the value obtained using GAP Version 10 using the following parameters: % identity using GAP Weight of 50 and Length Weight of 3%; similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.). The result of such calculations is referred to as the “sequence similarity” between two sequences.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1 ° C. to about 20° C. lower that the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman et al. (1970) J. Mol. Biol. 48:443. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

Disease and Pests

Compositions and methods for controlling pathogenic agents are provided. The anti-pathogenic compositions comprise maize peroxidase nucleotide and amino acid sequences. Particularly, the maize nucleic acid and amino acid sequences are selected from Zm-POX01, Zm-POX04, Zm-POX05, Zm-POX06, Zm-POX07, Zm-POX08, Zm-POX10, Zm-POX16, Zm-POX17, Zm-POX18, Zm-POX20, Zm-POX21, Zm-POX24, Zm-POX26, Zm-POX28, Zm-POX31, Zm-POX34, and Zm-POX37. Accordingly, the compositions and methods are also useful in protecting plants against fungal pathogens, viruses, nematodes, insects and the like.

By “disease resistance” or “pathogen resistance” is intended that the plants avoid the disease symptoms which are the outcome of plant-pathogen interactions. That is, pathogens are prevented from causing plant diseases and the associated disease symptoms, or alternatively, the disease symptoms caused by the pathogen is minimized or lessened. The methods of the invention can be utilized to protect plants from disease, particularly those diseases that are caused by plant pathogens. Examples of pathogens encompassed by the present invention include, but are not limited to, fungi, bacteria, viruses, other microbes, nematodes, and insects. Other examples include heat, drought, cold, reactive oxygen species, and radiation.

By “enhancing disease resistance” or “enhancing pathogen resistance” it is intended that the compositions of the invention are capable of suppressing, controlling, and/or killing the invading pathogenic organism. An antipathogenic composition of the invention will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

Assays that measure antipathogenic activity are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

In specific embodiments, methods for increasing pathogen resistance in a plant comprise stably transforming a plant with a DNA construct comprising an anti-pathogenic nucleotide sequence of the invention operably linked to promoter that drives expression in a plant. Such methods find use in agriculture particularly in limiting the impact of plant pathogens on crop plants. While the choice of promoter will depend on the desired timing and location of expression of the anti-pathogenic nucleotide sequences, preferred promoters include constitutive and pathogen-inducible promoters.

Additionally, the compositions can be used in formulations for their antimicrobial activities. The proteins of the invention can be formulated with an acceptable carrier into a pesticidal composition(s) that is for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

The compositions of the invention can be used for any application including coating surfaces to target microbes. In this manner, the target microbes include human pathogens or microorganisms. Surfaces that might be coated with the compositions of the invention include carpets and sterile medical facilities. Polymer bound polypeptides of the invention may be used to coat surfaces. Methods for incorporating compositions with antimicrobial properties into polymers are known in the art. See U.S. Pat. No. 5,847,047, herein incorporated by reference.

Additionally provided are transformed plants, plant cells, plant tissues and seeds thereof.

It is understood in the art that plant DNA viruses and fungal pathogens remodel the control of the host replication and gene expression machinery to accomplish their own replication and effective infection. The present invention may be useful in preventing such corruption of the cell.

The peroxidases of the invention function to mitigate or control the ROS burst seen in plants undergoing attack by a pathogen, generate secondary metabolites that may have antipathogenic activity and contribute to a plant's defense mechanism, and are required for lignin formation and cell wall strengthening. Hence, the peroxidase genes find use in disrupting cellular function of plant pathogens or insect pests as well as altering the defense mechanisms of a host plant to enhance resistance to disease or insect pests. While the invention is not bound by any particular mechanism of action to enhance disease resistance, the gene products, probably proteins or polypeptides, function to inhibit or prevent diseases in a plant.

The methods of the invention can be used with other methods available in the art for enhancing disease resistance in plants. For example, any one of a variety of second nucleotide sequences may be utilized, embodiments of the invention encompass those second nucleotide sequences that, when expressed in a plant, help to increase the resistance of a plant to pathogens. It is recognized that such second nucleotide sequences may be used in either the sense or antisense orientation depending on the desired outcome. Other plant defense proteins include those described in PCT patent publications WO 99/43823 and WO 99/43821, both of which are herein incorporated by reference.

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal and viral pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthephaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Broomrape, Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, lesion, and renniform nematodes, etc.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

Expression of Sequences

The nucleic acid sequences of the present invention can be expressed in a host cell such as bacteria, yeast, insect, mammalian, or preferably plant cells. It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

By “host cell” is meant a cell, which comprises a heterologous nucleic acid sequence of the invention. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The peroxidase sequences of the invention are provided in expression cassettes or DNA constructs for expression in the plant of interest. The cassette will include 5′ and 3′ regulatory sequences operably linked to a peroxidase sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the peroxidase sequence to be under the transcriptional regulation of the regulatory regions. The expression cassette may additionally contain selectable marker genes.

The expression cassette will include in the 5′-3′ direction of transcription, a transcriptional and translational initiation region, a peroxidase DNA sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence.

While it may be preferable to express the sequences using heterologous promoters, the native promoter sequences may be used. Such constructs would change expression levels of peroxidase in the host cell (i.e., plant or plant cell). Thus, the phenotype of the host cell (i.e., plant or plant cell) is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. Methods are available in the art for synthesizing plant-preferred genes. See, for example, U.S. Pat. Nos. 5,380,831, and 5,436,391, and Murray etal. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary MRNA structures.

The expression cassettes may additionally contain 5′ leader sequences in the expression cassette construct. Such leader sequences can act to enhance translation. Translation leaders are known in the art and include: picomavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 promoter and other constitutive promoters described in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. application Ser. No. 08/409,297), and the like. Other constitutive promoters include, for example, U.S. Pat. Nos. 5,608,149; 5,608,144; 5,604,121; 5,569,597; 5,466,785; 5,399,680; 5,268,463; and 5,608,142, and 6,177,611.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also the WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium verticillioides (previously Fusarium moniliforme). See, for example, Cordero et al. (1992) Physiol. Mol. Plant Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced peroxidase expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1 996) Plant Physiol. 112(3):1331-1341; Van Camp et al. (1996) Plant Physiol. 112(2):525-535; Canevascini et al. (1 996) Plant Physiol. 112(2):513 -524; Yamamoto et al. (1 994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1 993) Plant Mol. Biol. 23(6):1129-1138; and Matsuoka et al. (1993) Proc. Natl. Acad. Sci. USA 90(20):9586-9590.

The method of transformation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method, which provides for effective transformation/transfection may be employed.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat No. 5,563,055 and Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

The present invention may be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). Examples of selection markers for E. coli include, for example, genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al. (1983) Gene 22:229-235 and Mosbach et al. (1983) Nature 302:543-545).

A variety of eukaryotic expression systems such as yeast, insect cell lines, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention.

Synthesis of heterologous nucleotide sequences in yeast is well known. Sherman, F., et al. (1982) Methods in Yeast Genetics, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lists. The monitoring of the purification process can be accomplished by using Western blot techniques or radioimmunoassay of other standard immunoassay techniques.

The sequences of the present invention can also be ligated to various expression vectors for use in transfecting cell cultures of, for instance, mammalian, insect, or plant origin. Illustrative cell cultures useful for the production of the peptides are mammalian cells. A number of suitable host cell lines capable of expressing intact proteins have been developed in the art, and include the HEK293, BHK21, and CHO cell lines. Expression vectors for these cells can include expression control sequences, such as an origin of replication, a promoter (e.g. the CMV promoter, a HSV tk promoter or pgk (phosphoglycerate kinase) promoter), an enhancer (Queen et al. (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See, Schneider, J. Embryol. Exp. Morphol. 27:353-365 (1987).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al.(1983) J. Virol. 45:773-781). Additionally, gene sequences to control replication in the host cell may be incorporated into the vector such as those found in bovine papilloma virus type-vectors. Saveria-Campo, M., (1985) Bovine Papilloma Virus DNA a Eukaryotic Cloning Vector in DNA Cloning Vol. II a Practical Approach, D. M. Glover, Ed., IRL Press, Arlington, Va. pp. 213-238.

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art. Kuchler, R. J. (1997) Biochemical Methods in Cell Culture and Virology, Dowden, Hutchinson and Ross, Inc.

It is recognized that with these nucleotide sequences, antisense constructions, complementary to at least a portion of the messenger RNA (mRNA) for the peroxidase sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

In some embodiments, the content and/or composition of polypeptides of the present invention in a plant may be modulated by altering, in vivo or in vitro, the promoter of the nucleotide sequence to up- or down-regulate expression. For instance, an isolated nucleic acid comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art and discussed briefly, supra.

In general, concentration or composition is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds, which activate expression from these promoters, are well known in the art. In some embodiments, the polypeptides of the present invention are modulated in monocots, particularly maize.

Molecular Markers

The present invention provides a method of genotyping a plant comprising a polynucleotide of the present invention. Optionally, the plant is a monocot, such as maize or sorghum. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson 1996 (Chapter 2) in: Genome Mapping in plants (ed. Andrew H. Paterson) by Academic Press/R.G. Lands Company, Austin, Tex., pp. 7-21.

The particular method of genotyping in the present invention may employ any number of molecular marker analytic techniques such as, but not limited to, restriction fragment length polymorphism's (RFLPs). RFLPs are the product of allelic differences between DNA restriction fragments resulting from nucleotide sequence variability. As is well known to those of skill in the art, RFLPs are typically detected by extraction of genomic DNA and digestion with a restriction enzyme. Generally, the resulting fragments are separated according to size and hybridized with a probe; single copy probes are preferred. Restriction fragments from homologous chromosomes are revealed. Differences in fragment size among alleles represent an RFLP. Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, preferably within 20 or 10 cM, more preferably within 5, 3, 2, or 1 cM of a gene of the present invention.

In the present invention, the nucleic acid probes employed for molecular marker mapping of plant nuclear genomes selectively hybridize, under selective hybridization conditions, to a gene encoding a polynucleotide of the present invention. In some embodiments, the probes are selected from polynucleotides of the present invention. Typically, these probes are cDNA probes or restriction enzyme treated (e.g., PST I) genomic clones. The length of the probes is discussed in greater detail, supra, but is typically at least 15 bases in length, more preferably at least 20, 25, 30, 35, 40, or 50 bases in length. Generally, however, the probes are less than about 1 kilobase in length. Preferably, the probes are single copy probes that hybridize to a unique locus in haploid chromosome compliment. Some exemplary restriction enzymes employed in RFLP mapping are EcoRI, EcoRv, and SstI. As used herein the term “restriction enzyme” includes reference to a composition that recognizes and, alone or in conjunction with another composition, cleaves at a specific nucleotide sequence.

The method of detecting an RFLP comprises the steps of (a) digesting genomic DNA of a plant with a restriction enzyme; (b) hybridizing a nucleic acid probe, under selective hybridization conditions, to a sequence of a polynucleotide of the present of said genomic DNA; (c) detecting therefrom a RFLP. Other methods of differentiating polymorphic (allelic) variants of polynucleotides of the present invention can be had by utilizing molecular marker techniques well known to those of skill in the art including such techniques as: 1) single stranded conformation analysis (SSCA); 2)denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6)allele-specific PCR. Other approaches based on the detection of mismatches between the two complementary DNA strands include clamped denaturing gel electrophoresis (CDGE); heteroduplex analysis (HA); and chemical mismatch cleavage (CMC). Thus, the present invention further provides a method of genotyping comprising the steps of contacting, under stringent hybridization conditions, a sample suspected of comprising a polynucleotide of the present invention with a nucleic acid probe. Generally, the sample is a plant sample, preferably, a sample suspected of comprising a maize polynucleotide of the present invention (e.g., gene, mRNA). The nucleic acid probe selectively hybridizes, under stringent conditions, to a subsequence of a polynucleotide of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In preferred embodiments, the nucleic acid probe comprises a polynucleotide of the present invention.

The following examples are offered by way of illustration and not by way of limitation.

### EXPERIMENTAL

### Example 1

### Transformation and Regeneration of Transgenic Plants in Maize

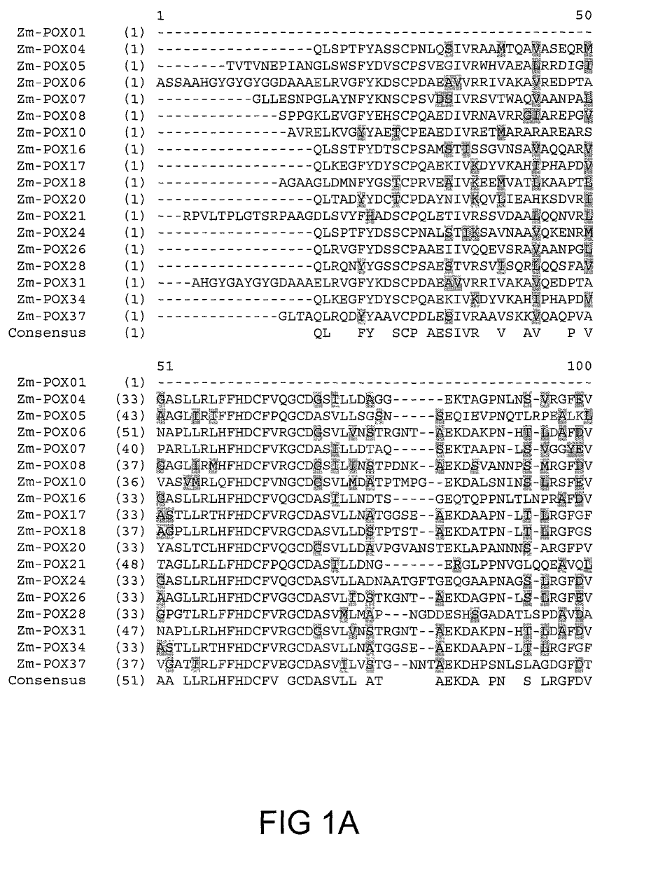

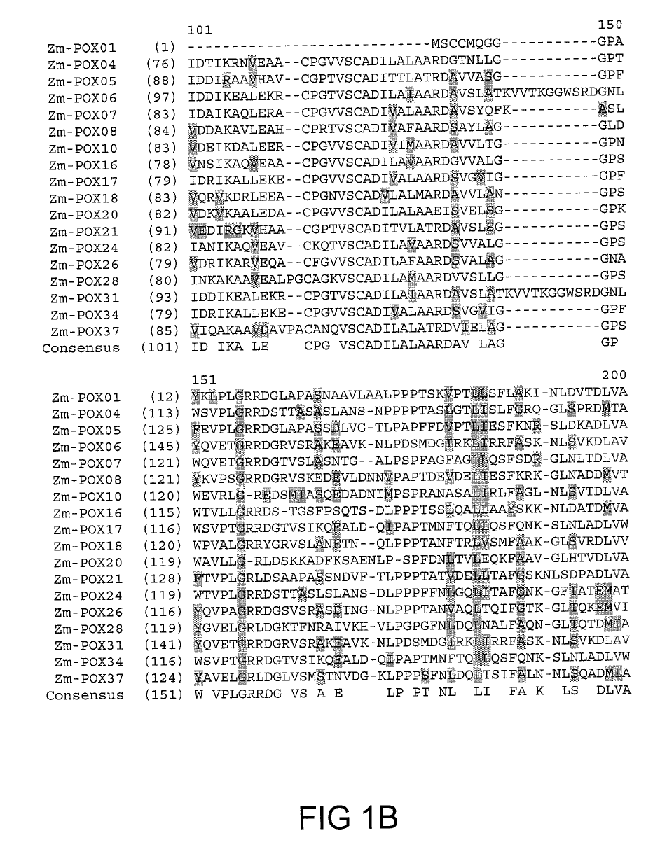

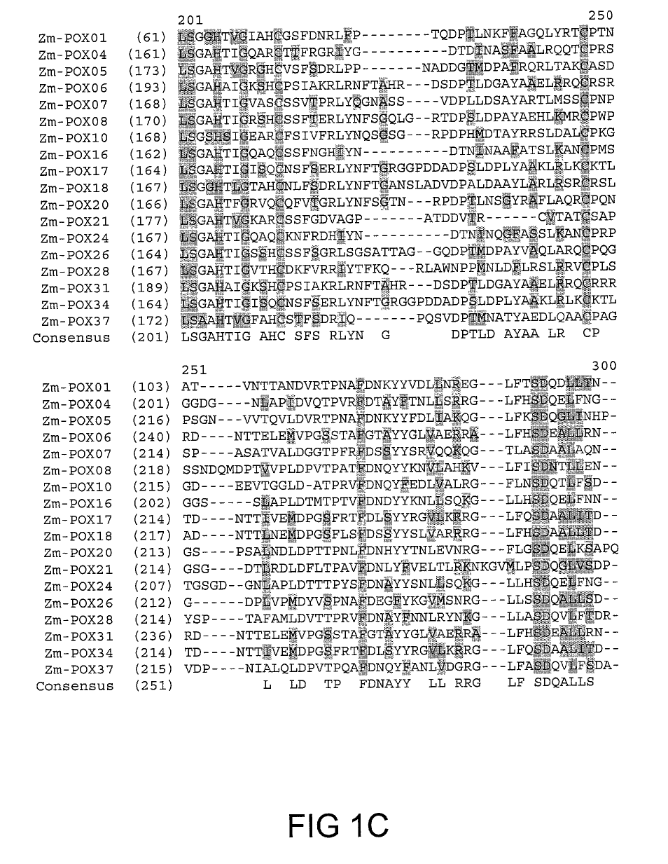

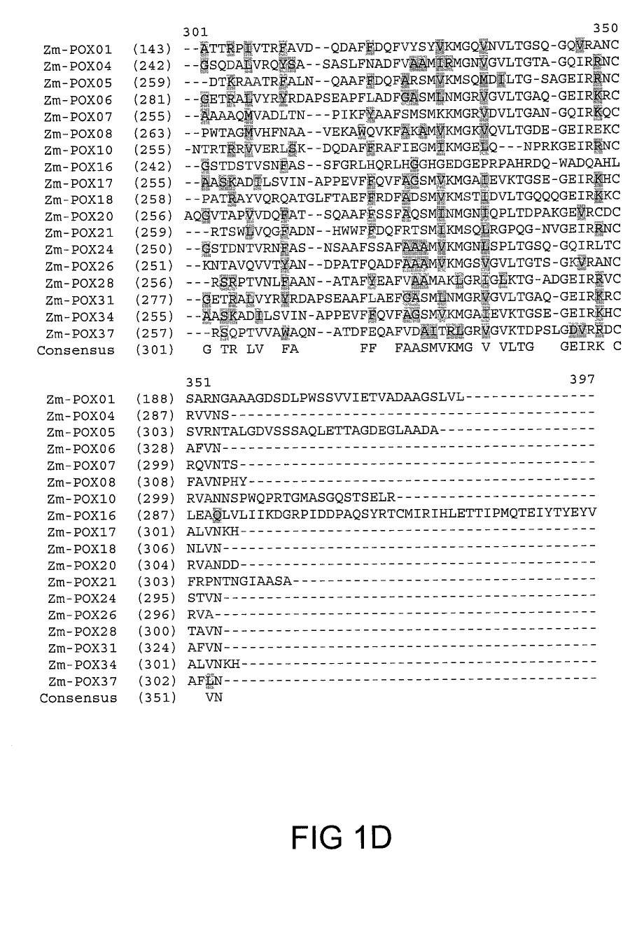

Immature maize embryos from greenhouse donor plants are bombarded with a plasmid containing a peroxidase nucleotide sequence operably linked to a ubiquitin promoter plus a plasmid containing the selectable marker gene PAT (Wohlleben et al. (1988) Gene 70:25-37) that confers resistance to the herbicide Bialaphos (FIG. 1). Transformation is performed as follows. All media recipes are in the Appendix.

Preparation of Target Tissue

The ears are surface sterilized in 30% Chlorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the peroxidase nucleotide sequence operably linked to a ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in TrisEDTA buffer (1 μg total)

100 μl 2.5 MCaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for altered defense response or altered GTPase activity.

Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1 511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 2

### Azrobacterium-mediated Transformation in Maize

For Agrobacterium-mediated transformation of maize with a peroxidase nucleotide sequence of the invention operably linked to a ubiquitin promoter, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the DNA construct containing the peroxidase nucleotide sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

### Example 3

### Soybean Embryo Transformation

Soybean embryos are bombarded with a plasmid containing the peroxidase nucleotide sequences operably linked to a ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 32 7:70-73, U.S. Pat. No. 4,945,050). A DuPont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the peroxidase nucleotide sequence operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μμ/gl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 4

### Sunflower Meristem Tissue Transformation

Sunflower meristem tissues are transformed with an expression cassette containing the peroxidase sequence operably linked to a ubiquitin promoter as follows (see also European Patent Number EP 0 486233, herein incorporated by reference, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al. (1990) Plant Cell Rep. 9: 55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant., 15: 473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6, and 8 g/l Phytagar.

The explants are subjected to microprojectile bombardment prior to Agrobacterium treatment (Bidney et al. (1992) Plant Mol. Biol. 18: 301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacteriun tumefaciens strain EHA105 is used in all transformation experiments. A binary plasmid vector comprising the expression cassette that contains the peroxidase gene operably linked to the ubiquitin promoter is introduced into Agrobacterium strain EHA105 via freeze-thawing as described by Holsters et al. (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for peroxidase-like activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of To plants (parental generation) maturing in the greenhouse are identified by NPTII ELISA and/or by peroxidase activity analysis of leaf extracts while transgenic seeds harvested from NPTII-positive To plants are identified by peroxidase activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 pl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium turmefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for peroxidase activity using assays known in the art. After positive (i.e., for peroxidase expression) explants are identified, those shoots that fail to exhibit peroxidase activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for peroxidase expression are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

### Example 5

### Sequence Analysis of the Maize Peroxidase Sequences

The Zm-POX1 cDNA (SEQ ID NO:1) is about 831 nucleotides in length with an open reading frame extending from about nucleotide 57 to 716. It encodes a 219 amino acid residue polypeptide (SEQ ID NO:2) with an approximate molecular weight of 23.2 kDa and a pI of about 5.6. The Zm-POX1 polypeptide (SEQ ID NO:2) shares sequence identity with peroxidases from adzuki bean (NCBI Accession No. JQ2252), barley (NCBI Accession No. S22505), and Arabidopsis thaliana (NCBI Accession Nos. CAA66962 and CAA67309).

The Zm-POX4 cDNA (SEQ ID NO:3) is about 1354 nucleotides in length with an open reading frame extending from about nucleotide 67 to 1008. It encodes a 313 amino acid residue polypeptide (SEQ ID NO:4). The mature polypeptide is predicted to have a length of about 291 amino acids, with a molecular weight of about 30.7 kDa and a pI of about 8.7. Zm-POX4 shares approximately 82.7% identity with a peroxidase from Cenchrus cilaris (NCBI accession No. AAA20472) as determined by the GAP algorithm described elsewhere herein using the default parameters. Zm-POX4 also shares sequence identity with peroxidases from Gossipyum hirsutum (NCBI Accession No. AAD43561), flax (NCBI Accession No. T08121), tobacco (NBI Accession Nos. AB027752 and BAA82306), and Scutellaria baicalensis (NCBI Accession No. BAA77389) The Zm-POX5 cDNA (SEQ ID NO:5) is about 1263 nucleotides in length with an open reading frame extending from about nucleotides 29 to 1099. It encodes a polypeptide (SEQ ID NO:6) of 356 amino acids. The mature polypeptide is predicted to have a length of 331 amino acid residues with a molecular weight of approximately 35.4 kDa and a pI of about 5.2. Zm-POX5 shares sequence identity with peroxidase polypeptides from Oryza sativa (NCBI Accession No. CAB53490), flax (NCBI Accession No. AAB02926), and Arabidopsis thaliana (NCBI Accession Nos. CAA67309 and CAA66962).

The Zm-POX6 cDNA (SEQ ID NO:7) is about 1519 nucleotides in length with an open reading frame extending from about nucleotides 146 to 1222. It encodes a 358 amino acid polypeptide (SEQ ID NO:8). The mature polypeptide is predicted to have a length of about 331 amino acids with an approximate molecular weight of 35.9 kDa and a pI of 9.2. Zm-POX6 shares sequence identity with peroxidase polypeptides from Oryza sativa (NCBI Accession No. P37834), Arabidopsis thaliana (NCBI Accession Nos. CAA66963, CAB89328, CAA67337, and CAA66965), spinach (NCBI Accession Nos. CAA76374 and T09218) and soybean (NCBI Accession No. AAD 11482).

The Zm-POX7 cDNA (SEQ ID NO:9) is about 1480 nucleotides in length with an open reading frame extending from about nucleotides 154 to 1194. It encodes a 346 amino acid polypeptide (SEQ ID NO:10). The mature polypeptide is predicted to have a length of about 304 amino acids with an approximate molecular weight of 31.9 kDa and a pI of 8.4. Zm-POX7 shares sequence identity with peroxidase polypeptides from tobacco (NCBI Accession Nos. T02962 and T02960), tomato (NCBI Accession No. S51584), and spinach (NCBI Accession No. CAA76374).

The Zm-POX8 cDNA (SEQ ID NO: 11) is about 1183 nucleotides in length with an open reading frame extending from about nucleotides 60 to 1079. It encodes a 339 amino acid polypeptide (SEQ ID NO:12). The mature polypeptide is predicted to have a length of about 314 amino acids with an approximate molecular weight of 34.5 kDa and a pI of about 5.6. Zm-POX8 shares sequence identity with peroxidase polypeptides from Scutellaria baicalensis (NCBI Accession No. BAA77387), spinach (NCBI Accession No.AAF63024), and Arabidopsis thaliana (NCBI Accession Nos. T1 3020 and CAA67337).

The Zm-POX10 cDNA (SEQ ID NO: 13) is about 1407 nucleotides in length with an open reading frame extending from about nucleotides 142 to 1185. It encodes a 347 amino acid polypeptide (SEQ ID NO:14). The mature polypeptide is predicted to have a length of about 322 amino acids with an approximate molecular weight of 35.5 kDa and a pI of about 5.2. Zm-POX10 shares sequence identity with peroxidase polypeptides from Arabidopsis thaliana (NCBI Accession No. CAA67092), and Populus balsamifera subsp. trichocarpa (NCBI Accession No.CAA66037).

The Zm-POX16 cDNA (SEQ ID NO:16) is about 1388 nucleotides in length with an open reading frame extending from about nucleotides 87 to 1175. It encodes a 362 amino acid polypeptide (SEQ ID NO:17). The mature polypeptide is predicted to have a length of about 333 amino acids with an approximate molecular weight of 35.7 kDa and a pI of about 5.2. The nucleotide sequence was isolated in a form containing an unspliced intron. This nucleotide sequence is shown in SEQ ID NO:15, with the intron extending from about nucleotide 315 to 491. Zm-POX16 shares sequence identity with peroxidase polypeptides from oat (NCBI Accession Nos. AAC31550 and AAC31551), Oryza sativa (NCBI Accession Nos.P37835, AAC49821, and S22087), and wheat (NCBI Accession No. S61405).

The Zm-POX17 cDNA (SEQ ID NO:18) is about 1467 nucleotides in length with an open reading frame extending from about nucleotides 109 to 1095. It encodes a 328 amino acid polypeptide (SEQ ID NO:19). The mature polypeptide is predicted to have a length of about 306 amino acids with an approximate molecular weight of 33.2 kDa and a pI of about 6.4. Zm-POX17 shares sequence identity with peroxidase polypeptides from tomato (NCBI Accession Nos. AAA65637 and S51584), spinach (NCBI Accession Nos. CAA76374 and T09218), soybean (NCBI Accession Nos. AAD11481, AAD11482) and Arabidopsis thaliana (NCBI Accession Nos. CAA66965 and CAA67360).

The Zm-POX1 8 cDNA (SEQ ID NO:20) is about 1522 nucleotides in length with an open reading frame extending from about nucleotides 187 to 1170. It encodes a 327 amino acid polypeptide (SEQ ID NO:21). The mature polypeptide is predicted to have a length of about 309 amino acids with an approximate molecular weight of 33.4 kDa and a pI of about 7.7. Zm-POX18 shares sequence identity with peroxidase polypeptides from Oryza sativa (NCBI Accession No. P37834), Arabidopsis thaliana (NCBI Accession Nos. CAA66963, CAA66965, and CAA67360), Spirodela polyrrhiza (NCBI Accession Nos. S40268), tomato (NCBI Accession No. AAA65637) and spinach (NCBI Accession No. CAA76374).

The Zm-POX20 cDNA (SEQ ID NO:22) is about 1451 nucleotides in length with an open reading frame extending from about nucleotides 170 to 1198. It encodes a 342 amino acid polypeptide (SEQ ID NO:23). The mature polypeptide is predicted to have a length of about 309 amino acids with an approximate molecular weight of 33.2 kDa and a pI of about 4.9. Zm-POX20 shares sequence identity with peroxidase polypeptides from Phaseolus vulgaris (NCBI Accession No. AAD37430), Populus sieboldii x Populus grandidentata (NCBI Accession No. S60054), sweet potato (NCBI Accession No. CAB94692), Populus balsamifera subsp. trichocarpa (NCBI Accession No. CAA66037) and horseradish (NCBI Accession No. P80679), Arabidopsis thaliana (NCBI Accession Nos. CAA68212 and T02507), and Populus kitakamien (NCBI Accession No. BAA06335).

The Zm-POX21 cDNA (SEQ ID NO:24) is about 1334 nucleotides in length with an open reading frame extending from about nucleotides 62 to 1075. It encodes a 337 amino acid polypeptide (SEQ ID NO:25). The mature polypeptide is predicted to have a length of about 314 amino acids with an approximate molecular weight of 33.2 kDa and a pI of about 5.2. Zm-POX21 shares sequence identity with peroxidase polypeptides from Oryza sativa (NCBI Accession Nos. CAB53489, CAB53485, CAB53488, CAB53490, and CAB53486), parsley (NCBI Accession No. S55035), and adzuki bean (NCBI Accession No. JQ2252).

The Zm-POX24 cDNA (SEQ ID NO:26) is about 1285 nucleotides in length with an open reading frame extending from about nucleotides 96 to 1058. It encodes a 320 amino acid polypeptide (SEQ ID NO:27). The mature polypeptide is predicted to have a length of about 298 amino acids with an approximate molecular weight of 31.1 kDa and a pI of about 6.2. Zm-POX24 shares sequence identity with peroxidase polypeptides from Cenchrus ciliaris (NCBI Accession No. AAA20473) Oryza sativa (NCBI Accession Nos. AAC49819, P37835, AAC49821, S22087, AAC49818, and AAC49820), wheat (NCBI Accession Nos. S61408, S61405, S61406, S13375, and Q05855), Avena sativa (NCBI Accession No. AAC31550), and barley (NCBI Accession Nos. T06172 and P27337).

The Zm-POX26 cDNA (SEQ ID NO:28) is about 1159 nucleotides in length with an open reading frame extending from about nucleotides 7 to 969. It encodes a 320 amino acid polypeptide (SEQ ID NO:29). The mature polypeptide is predicted to have a length of about 298 amino acids with an approximate molecular weight of 30.8 kDa and a pI of about 6.1. Zm-POX26 shares sequence identity with peroxidase polypeptides from Scutellaria baicalensis (NCBI Accession No. BAA77387), spinach (NCBI Accession No. AAF63024), Oryza sativa (NCBI Accession No. P37834), Gossypium hirsutum (NCBI Accession No. AAD43561), peanut (NCBI Accession Nos. P22195 and A38265), Arabidopsis thaliana (NCBI Accession No. T13020), Spirodela polyrrhiza (NCBI Accession No. S40268), and wheat (NCBI Accession No. S61408).

The Zm-POX28 cDNA (SEQ ID NO:30) is about 1310 nucleotides in length with an open reading frame extending from about nucleotidesl00 to 1098. It encodes a 332 amino acid polypeptide (SEQ ID NO:3 1). The mature polypeptide is predicted to have a length of about 303 amino acids with an approximate molecular weight of 32.8 kDa and a pI of about 9.1. Zm-POX28 shares sequence identity with peroxidase polypeptides from Arabidopsis thaliana (NCBI Accession Nos. CAA70034, T01626, T14077, T04709, T04710, CAA67362, and CAA66967), white clover (NCBI Accession No. CAA09881), and alfalfa (NCBI Accession No. JC4782).

The Zm-POX31 cDNA (SEQ ID NO:32) is about 1170 nucleotides in length with an open reading frame extending from about nucleotides25 to 1092. It encodes a 355 amino acid polypeptide (SEQ ID NO:33). The mature polypeptide is predicted to have a length of about 327 amino acids with an approximate molecular weight of 35.6 kDa and a pI of about 9.2. Zm-POX31 shares sequence identity with peroxidase polypeptides from Arabidopsis thaliana (NCBI Accession Nos. CAA66963, CAA67337, and T13020), Oryza sativa (NCBI Accession No. P37834), spinach (NCBI Accession Nos. CAA76374 and T09218), and soybean (NCBI Accession No. AAD1 1482).

The Zm-POX34 cDNA (SEQ ID NO:34) is about 1391 nucleotides in length with an open reading frame extending from about nucleotides103 to 1089. It encodes a 328 amino acid polypeptide (SEQ ID NO:35). The mature polypeptide is predicted to have a length of about 306 amino acids with an approximate molecular weight of 33.2 kDa and a pI of about 6.4. Zm-POX34 shares sequence identity with peroxidase polypeptides from tomato (NCBI Accession Nos. AAA65637 and S51584), spinach (NCBI Accession Nos. CAA76374 and T09218), soybean (NCBI Accession Nos. AAD1 1481 and AAD1 1482), and Arbidopsis thaliana (NCBI Accession No. CAA66965 and CAA67360).

The Zm-POX37 cDNA (SEQ ID NO:36) is about 1476 nucleotides in length with an open reading frame extending from about nucleotides 259 to 1236. It encodes a 325 amino acid polypeptide (SEQ ID NO:37). The mature polypeptide is predicted to have a length of about 305 amino acids with an approximate molecular weight of 32.2 kDa and a pI of about 4.4. Zm-POX34 shares sequence identity with peroxidase polypeptides from alfalfa (NCBI Accession Nos. JC4782 and T09667), Arabidopsis thaliana (NCBI Accession Nos. CAA67339, T04709, CAA70034, CAA66967, and T04710), and white clover (NCBI Accession No. CAA09881).

### Example 6

### Peroxidase Gene Expression in Response to C. carbonum Toxin

The HC toxin produced by the pathogenic fungus C. carbonum has been shown to be an in vitro and in vivo regulator of histone deacetylase. Inhibition of histone deacetylase may prevent effective host responses by influencing chromatin structure at defense response-related promoters. Most maize inbred lines produce a toxin reductase that confers resistance to the fungus C. carbonum, but this enzyme is not present in genotypes Pr (described in Multani et al. (1998), Proc. Natl. Acad. Sci. USA 95:1686-1691, herein incorporated by reference) and A188. When a C. carbonum strain that lacks the ability to synthesize HC-toxin is applied to Pr plants, the result is an incompatible reaction; i.e. the formation of limited lesions with no progression to disease. If the fungus is applied to the Pr plant along with exogenous toxin, however, the result is compatibility, i.e. disease lesions form on leaves and mold forms on ears. Thus, a comparison of the expression of a gene in the presence of toxin positive and toxin negative C. carbonum strains allows for the elucidation of toxin-induced versus defense-induced gene expression.

Microarray hybridization was used to determine the expression levels of the novel maize peroxidase nucleotide sequences of the invention in 20-day old Pr, A188 and A63 maize lines exposed to the following treatments: (1) control (water only), (2) C. carbonum toxin⁻, (3) HC-toxin, (4) ) C. carbonum toxin⁻+HC-toxin, and (5) C. carbonum toxin⁺. C. carbonum conidia (1×10⁵ per ml) and HC toxin (1 μg/ml) were applied by spraying to run-off in a dilute solution of TWEEN-20. The flats were bagged and kept at high humidity in a 27° C. incubator. Samples were collected 3, 6, and 22 hours following the treatment. Three independent replicates were performed for each treatment.

In an alternative inoculation method, the same treatments were applied by soaking 3MM filter paper sections and sealing them to the leaf surfaces.

Zm-POX24 expression levels were increased 6.4 fold in leaves treated for 6 hours with C. carbonum toxin⁻ in comparison with control leaves, confirming the defense-related expression of this gene.

In another approach, expression levels of the peroxidase nucleotide sequences were measured in suspension cell cultures, which provide a large number of uniformly treated cells. The cells were derived from a K61×B73 cross, which is sensitive to Hc-toxin. In these experiments, chitooligosaccharide (0.1 mg/ml), a component of fungal cell walls commonly used as an elicitor of the plant defense response, was used in place of fungal spores to elicit the defense response in the absence or presence of 1 μg/ml HC-toxin. Duplicate cell samples were harvested 2 hr. after treatment, with the exception of one set of cultures, which was given a 2 hr. pre-treatment with HC-toxin prior to the addition of chitooligosacchride for 2 hours.

Both Zm-POX08 and Zm-POX01 showed defense-induced expression in this experiment, with Zm-POX08 message levels increasing 14.1 fold and Zm-POX01 message levels increasing 3.7 fold after a two hour treatment with chitooligosaccharide. Up-regulation of Zm-POX08 message was also observed after a one hour treatment of GS3 cells with chitooligosaccharaides.

### Example 7

### Peroxidase Gene Expression in Response to F. monilforme

Fusarium verticillioides (previously F. moniliforme) is a fungal pathogen of maize which causes ear mold and stalk rot. The expression of the novel peroxidase sequences after two or six hours of treatment with F. verticillioides spores or with chitooligosaccharide was determined by microarray hybridization. In these experiments, Zm-POX 5 message levels increase 5.5 fold after two hours, and 6.1 fold after six hours of treatment with F. verticillioides spores, indicating that expression of this gene is induced in the defense response. Zm-POX37 levels decreased 2.5 fold after two hours of treatment with F. verticillioides spores, and decreased 2.1 fold after two hours of treatment with chitooligosaccharide, indicating that the expression of this gene is also regulated by the defense response.

### Example 8

### Identification of a Maize Peroxidase Sequence Induced by the Defense Response

Differential gene expression profiling was used to identify maize genes that were elicited during the defense response. A nucleotide sequence having 96% identity to nucleotides 748-1054 of Zm-POX4 (SEQ ID NO:3) was isolated in this experiment, indicating that Zm-POX4 is induced by the defense response. The levels of this nucleotide sequence increased approximately five fold in the defense response.

### Example 9

### Expression of Maize Peroxidase Polypeptides in Response to Infection by Cochlibolus heterostrophus (Bipolaris maydis)

The expression patterns of peroxidase isozymes was measured in wildtype and four rhm1 allele mutant (B. maydis resistant) strains of maize in the absence or presence of B. maydis infection using isoelectric focussing. In the uninfected control tissues, four anionic peroxidase species were expressed, and there were no consistent differences in the level or type of peroxidase expressed in the rhml mutants versus the wildtype strains. Following infection with B. maydis, the pattern of peroxidases was more complex, with at least six species being expressed including the four expressed in the absence of infection. The levels of all of the peroxidase polypeptides increased following infections, with levels of the three most anionic species increasing the most.

Methods

For the peroxidase isozyme expression assay, 100 μg of ground powder for each sample was extracted in 1 ml of pH 7.4, 0.1 M sodium phosphate buffer, followed by centrifugation at 10,000 g for 15 minutes. The supernatants (15 μl) for these samples were subjected to isoelectric focusing electrophoresis essentially as described in Dowd, P. F.(1994) J. Chem. Ecol. 20(11):2777-2803. The gels were pre-cast wide range (pH 3.5-9.5) polyacrylamide gels (Pharmacia Biotech, Piscataway, N.J.). Electrophoresis was performed at 25W for 1.5 hours. Peroxidase activity was visualized essentially as described (Dowd, supra). The gel with visible peroxidase bands was photographed using a Sigma T1203 trans-illuminator and a 35 mM camera.

### Example 10

### Expression of Maize Peroxidase Polypeptides in Response to avrRxv Gene Expresion

The expression patterns of peroxidase isozymes was measured in callus in the absence of presence of ERE-driven avrRxv gene expression. As a control, western blot analysis was used to assay changes in the expression of chitinase and PR1, two frequently used makers for plant defense activation. Levels of both chitinase and PR1 incrase in ERE-avrRxv transfected callus treated with estradiol in comparison with the levels of these proteins in untreated callus. Two different callus lines, 197 and 186, were used for this analysis. Line 186 showed induction of chitinase and increased expression of Pr1 following estradiol treatment. Line 186 showed slower growth and general browning in comparison to line 197, possibly due to a lower level of avrRxv expression.

The expression of peroxidase isozymes was measured in the absence or presence of avrRxv gene expression using methods described elsewhere herein. Little avrRxv-induced change was observed in the expression of anionic peroxidase species for both lines 197 and 186. However, there was a marked increase in the expression of cationic peroxidase species in both lines. The induction of cationic peroxidase species by avrRxv is of special note, because a cationic peroxidase has been shown to be induced in compatible resistance interactions between rice and Xanthamosas oryzae pv oryzae (Reimers et al. (1992) Plant Physiol. 99:1044-1050).

### Example 11

### Chromosomal Location of Novel Maize Peroxidase Genes

A number of disease-related gene loci and quantitative trait loci (QTL's) for various traits, including disease resistance, are known in maize. Consequently, it was of interest to map the novel peroxidase genes of the present invention to their chromosomal locations. The chromosomal locations of the maize peroxidase gene of the present invention are given Table I. Table II gives the map positions of a number of know maize disease resistance loci and QTL's in relation to the position of the peroxidase genes of the invention.

TABLE I

| Gene Name | Map Position |
|---|---|
| Zm-POX01 | 3.04 |
| Zm-POX04 | 9.03/9.04 |
| Zm-POX05 | 2.04 |
| Zm-POX06 | 1.09 |
| Zm-POX07 | 3.03 |
| Zm-POX08 | 10.04 |
| Zm-POX10 | 7.02 |
| Zm-POX16 | 7.06 |
| Zm-POX17 | 5.04 |
| Zm-POX20 | 5.04 |
| Zm-POX24 | 7.06 |
| Zm-POX26 | 1.03 |
| Zm-POX28 | 2.05 |
| Zm-POX31 | 1.09 |
| Zm-POX34 | 5.04 |
| Zm-POX37 | 1.1 |

TABLE II

| Map Position | Disease Trait/Peroxidase Gene |
|---|---|
| 1.01 | European Corn Borer QTL |
| 1.01/1.02 | Northern Corn Leaf Blight QTL |
| 1.03 | Zm-POX26 |
| 1.04 | Gray Leaf Spot QTL |
| 1.04 | Maize Streak Virus (msv1) |
| 1.03/1.06 | Northern Corn Leaf Blight QTL |
| 1.05 | Stewart's Wilt QTL |
| 1.09 | Zm-POX06 |
| 1.09 | Zm-POX31 |
| 1.10 | Zm-POX37 |
| 2.04 | Lesion Mimic Les1 |
| 2.04 | Lesion Mimic Les15 |
| 2.04 | Zm-Pox05 |
| 2.04/2.05 | Gray Leaf Spot QTL |
| 2.05 | Zm-POX28 |
| 3.03 | Zm-POX07 |
| 3.04 | Zm-POX01 |
| 3.04 | rp3 Rust Resistance |
| 3.04/3.05 | European Corn Borer QTL |
| 3.04/3.05 | Gibberella Stalk Rot QTL |
| 3.07/3.08 | Northern Corn Leaf Blight QTL |
| 5.04 | Gibberella Stalk Rot QTL |
| 5.04 | Zm-POX17 |
| 5.04 | Zm-POX20 |
| 5.04 | Zm-POX34 |
| 5.06 | Northern Corn Leaf Blight QTL |
| 6.01 | Southern Corn Leaf Blight (rhm1) |
| 7.02 | Zm-POX10 |
| 7.06 | Zm-POX16 |
| 7.06 | Zm-POX24 |
| 9.03/9.04 | Zm-POX04 |
| 9.05 | Southwestern Corn Borer QTL |
| 10.01 | Rust resistance (Puccinia sorghi) |
| 10.04 | Zm-POX08 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated polypeptide comprising an amino acid sequence selected from the group consisting of:
(a) a polypeptide sequence comprising the amino acid sequence set forth in SEQ ID NO:2, 4, 6, 8, 10, 12, 14, 17, 19, 21, 23, 25, 27, 29, 31, 33, 35, or 37;
(b) a polypeptide having at least 80% sequence identity with at least one of the sequences of (a), wherein said polypeptide retains peroxidase-like activity;
(c) a polypeptide encoded by a nucleotide sequence that hybridizes under stringent conditions to at least one nucleotide sequence selected from the group consisting of SEQ ID NOS:1, 3, 5, 7, 9, 11, 13, 15, 16, 18, 20, 22, 24, 26, 28, 30, 32, 34, and 36; and
(d) a fragment comprising at least 20 contiguous amino acids of at least one of the amino acid sequences set forth in SEQ ID NO:2, 4, 6, 8, 10, 12, 14, 17, 19, 21, 23, 25, 27, 29, 31, 33, 35, or 37, wherein said fragment has peroxidase-like activity.

2. An isolated nucleic acid molecule comprising a nucleotide sequence selected from the group consisting of:
(a) the nucleotide sequence set forth in SEQ ID NO:1, 3, 5, 7, 9, 11, 13, 15, 16, 18, 20, 22, 24, 26, 28, 30, 32, 34, or 36;
(b) a nucleotide sequence encoding the amino acid sequence set forth in SEQ ID NO:2, 4, 6, 8, 10, 12, 14, 17, 19, 21, 23, 25, 27, 29, 31, 33, 35, or 37;
(c) a nucleotide sequence comprising at least 16 contiguous nucleotides of a nucleotide sequence of (a) or (b), wherein said sequence encodes a polypeptide having peroxidase-like activity;
(d) a nucleotide sequence having at least 60% sequence identity with at least one nucleotide sequence of (a) or (b), wherein said nucleotide sequence having at least 60% sequence identity with at least one nucleotide sequence of (a) or (b) encodes a polypeptide having peroxidase-like activity;
(e) a nucleotide sequence that hybridizes under stringent conditions to at least one nucleotide sequence of (a) or (b), wherein said nucleotide sequence that hybridizes under stringent conditions to at least one nucleotide sequence of (a) or (b) encodes a polypeptide having peroxidase-like activity; and
(f) a nucleotide sequence complementary to a nucleotide sequence of (a), (b), (c), (d), or (e).

3. The nucleic acid molecule of claim 2, wherein said nucleotide sequence is operably linked to a promoter that drives expression in a plant cell.

4. An expression vector comprising the nucleic acid molecule of claim 3.

5. A host cell having stably incorporated into its genome at least one nucleotide sequence, wherein said nucleotide sequence is operably linked to a heterologous promoter that drives expression in the host cell, wherein said nucleotide sequence is selected from the group consisting of:
(a) the nucleotide sequence set forth in SEQ ID NO:1, 3, 5, 7, 9, 11, 13, 15, 16, 18, 20, 22, 24, 26, 28, 30, 32, 34, or 36;
(b) a nucleotide sequence encoding the amino acid sequence set forth in SEQ ID NO:2, 4, 6, 8, 10, 12, 14, 17, 19, 21, 23, 25, 27, 29, 31, 33, 35, or 37;
(c) a nucleotide sequence comprising at least 16 contiguous nucleotides of a nucleotide sequence of (a) or (b), wherein said sequence encodes a polypeptide having peroxidase-like activity;
(d) a nucleotide sequence having at least 60% sequence identity with at least one nucleotide sequence of (a) or (b), wherein said nucleotide sequence having at least 60% sequence identity with at least one nucleotide sequence of (a) or (b) encodes a polypeptide having peroxidase-like activity;
(e) a nucleotide sequence that hybridizes under stringent conditions to at least one nucleotide sequence of (a) or (b), wherein said nucleotide sequence that hybridizes under stringent conditions to at least one nucleotide sequence of (a) or (b) encodes a polypeptide having peroxidase-like activity; and
(f) a nucleotide sequence complementary to a nucleotide sequence of (a), (b), (c), (d), or (e).

6. The host cell of claim 5, wherein said cell is a plant cell.

7. A plant having stably incorporated into its genome at least one nucleotide sequence operably linked to a heterologous promoter that drives expression in a plant cell, wherein said nucleotide sequence is selected from the group consisting of:
(a) the nucleotide sequence set forth in SEQ ID NO:1, 3, 5, 7, 9, 11, 13, 15, 16, 18, 20, 22, 24, 26, 28, 30, 32, 34, or 36;
(b) a nucleotide sequence encoding the amino acid sequence set forth in SEQ ID NO:2, 4, 6, 8, 10, 12, 14, 17, 19, 21, 23, 25, 27, 29, 31, 33, 35, or 37;
(c) a nucleotide sequence comprising at least 16 contiguous nucleotides of a nucleotide sequence of (a) or (b), wherein said sequence encodes a polypeptide having peroxidase-like activity;
(d) a nucleotide sequence having at least 60% sequence identity with at least one nucleotide sequence of (a) or (b), wherein said nucleotide sequence having at least 60% sequence identity with at least one nucleotide sequence of (a) or (b) encodes a polypeptide having peroxidase-like activity;
(e) a nucleotide sequence that hybridizes under stringent conditions to at least one nucleotide sequence of (a) or (b), wherein said nucleotide sequence that hybridizes under stringent conditions to at least one nucleotide sequence of (a) or (b) encodes a polypeptide having peroxidase-like activity; and
(f) a nucleotide sequence complementary to a nucleotide sequence of (a), (b), (c), (d), or (e).

8. The plant of claim 7, wherein said promoter is a constitutive promoter.

9. The plant of claim 7, wherein said promoter is a tissue-preferred promoter.

10. The plant of claim 7, wherein said promoter is an inducible promoter.

11. The plant of claim 10, wherein said promoter is a pathogen-inducible promoter.

12. The plant of claim 7, wherein said plant is a monocot.

13. The plant of claim 12, wherein said monocot is maize, rice, or wheat.

14. The plant of claim 12, wherein said monocot is maize.

15. The plant of claim 7, wherein said plant is a dicot.

16. The plant of claim 15, wherein said dicot is soybean or sunflower.

17. The transformed seed of the plant of claim 7.

18. A method for enhancing the defense response in a plant, said method comprising stably incorporating into the genome of said plant at least one nucleotide sequence operably linked to a heterologous promoter that drives expression in a plant cell, wherein said nucleotide sequence is selected from the group consisting of:
(a) the nucleotide sequence set forth in SEQ ID NO:3, 7, 9, 20, 30, or 32;
(b) a nucleotide sequence encoding the amino acid sequence set forth in SEQ ID NO:4, 8, 10, 21, 31, or 33;
(c) a nucleotide sequence comprising at least 16 contiguous nucleotides of a nucleotide sequence of (a) or (b), wherein said sequence encodes a polypeptide having peroxidase-like activity;
(d) a nucleotide sequence having at least 60% sequence identity with at least one nucleotide sequence of (a) or (b), wherein said nucleotide sequence having at least 60% sequence identity with at least one nucleotide sequence of (a) or (b) encodes a polypeptide having peroxidase-like activity;
(e) a nucleotide sequence that hybridizes under stringent conditions to at least one nucleotide sequence of (a) or (b), wherein said nucleotide sequence that hybridizes under stringent conditions to at least one nucleotide sequence of (a) or (b) encodes a polypeptide having peroxidase-like activity; and
(f) a nucleotide sequence complementary to a nucleotide sequence of (a), (b), (c), (d), or (e).

19. The method of claim 18, wherein said promoter is a constitutive promoter.

20. The method of claim 18, wherein said promoter is a pathogen inducible promoter.

21. The method of claim 18, wherein said plant is a monocot.

22. The method of claim 21, wherein said monocot is maize, wheat, or rice.

23. The method of claim 18, wherein said plant is a dicot.

24. The method of claim 23, wherein said dicot is soybean or sunflower.

25. A method for enhancing the defense response in a plant, said method comprising stably incorporating into the genome of said plant at least one nucleotide sequence operably linked to a heterologous promoter that drives expression in a plant cell, wherein said defense response does not include a response to an insect, and wherein said nucleotide sequence is selected from the group consisting of:
(a) the nucleotide sequence set forth in SEQ ID NO:1, 5, 11, 13, 16, 18, 20, 22, 24, 26, 34, or 36;
(b) a nucleotide sequence encoding the amino acid sequence set forth in SEQ ID NO:2, 6, 12, 14, 17, 19, 23, 25, 27, 29, 35, or 37;
(c) a nucleotide sequence comprising at least 16 contiguous nucleotides of a nucleotide sequence of (a) or (b), wherein said sequence encodes a polypeptide having peroxidase-like activity;
(d) a nucleotide sequence having at least 60% sequence identity with at least one nucleotide sequence of (a) or (b), wherein said nucleotide sequence having at least 60% sequence identity with at least one nucleotide sequence of (a) or (b) encodes a polypeptide having peroxidase-like activity;
(e) a nucleotide sequence that hybridizes under stringent conditions to at least one nucleotide sequence of (a) or (b), wherein said nucleotide sequence that hybridizes under stringent conditions to at least one nucleotide sequence of (a) or (b) encodes a polypeptide having peroxidase-like activity; and
(f) a nucleotide sequence complementary to a nucleotide sequence of (a), (b), (c), (d), or (e).

26. The method of claim 25, wherein said promoter is a constitutive promoter.

27. The method of claim 25, wherein said promoter is a pathogen inducible promoter.

28. The method of claim 25, wherein said plant is a monocot.

29. The method of claim 28, wherein said monocot is maize, wheat, or rice.

30. The method of claim 25 wherein said plant is a dicot.

31. The method of claim 30, wherein said dicot is soybean or sunflower.

32. A method for enhancing stalk strength in a plant, said method comprising stably incorporating into the genome of said plant at least one nucleotide sequence operably linked to a heterologous promoter that drives expression in a plant cell, wherein said nucleotide sequence is selected from the group consisting of:
(a) the nucleotide sequence set forth in SEQ ID NO:1, 3, 5, 7, 9, 11, 13, 15, 16, 18, 20, 22, 24, 26, 28, 30, 32, 34, or 36;
(b) a nucleotide sequence encoding the amino acid sequence set forth in SEQ ID NO:2, 4, 6, 8, 10, 12, 14, 17, 19,21,23,25,27,29,31, 33,35, or 37;
(c) a nucleotide sequence comprising at least 16 contiguous nucleotides of a nucleotide sequence of (a) or (b), wherein said sequence encodes a polypeptide having peroxidase-like activity;
(d) a nucleotide sequence having at least 60% sequence identity with at least one nucleotide sequence of (a) or (b), wherein said nucleotide sequence having at least 60% sequence identity with at least one nucleotide sequence of (a) or (b) encodes a polypeptide having peroxidase-like activity;
(e) a nucleotide sequence that hybridizes under stringent conditions to at least one nucleotide sequence of (a) or (b), wherein said nucleotide sequence that hybridizes under stringent conditions to at least one nucleotide sequence of (a) or (b) encodes a polypeptide having peroxidase-like activity; and
(f) a nucleotide sequence complementary to a nucleotide sequence of (a), (b), (c), (d), or (e).

33. The method of claim 32, wherein said promoter is a constitutive promoter.

34. The method of claim 32, wherein said promoter is a pathogen inducible promoter.

35. The method of claim 32, wherein said plant is a monocot.

36. The plant of claim 35, wherein said monocot is maize, wheat, or rice.

37. The method of claim 32, wherein said plant is a dicot.

38. The method of claim 37, wherein said dicot is soybean or sunflower.

39. A method for preventing oxidative damage following anoxia in a plant, said method comprising stably incorporating into the genome of said plant at least one nucleotide sequence operably linked to a heterologous promoter that drives expression in a plant cell, wherein said nucleotide sequence is selected from the group consisting of:
(a) the nucleotide sequence set forth in SEQ ID NO:1, 3, 5, 7, 9, 11, 13, 15, 16, 18, 20, 22, 24, 26, 28, 30, 32, 34, or 36;
(b) a nucleotide sequence encoding the amino acid sequence set forth in SEQ ID NO:2, 4, 6, 8, 10, 12, 14, 17, 19, 21, 23, 25, 27, 29, 31, 33, 35, or 37;
(c) a nucleotide sequence comprising at least 16 contiguous nucleotides of a nucleotide sequence of (a) or (b), wherein said sequence encodes a polypeptide having peroxidase-like activity;
(d) a nucleotide sequence having at least 60% sequence identity with at least one nucleotide sequence of (a) or (b), wherein said nucleotide sequence having at least 60% sequence identity with at least one nucleotide sequence of (a) or (b) encodes a polypeptide having peroxidase-like activity;
(e) a nucleotide sequence that hybridizes under stringent conditions to at least one nucleotide sequence of (a) or (b), wherein said nucleotide sequence that hybridizes under stringent conditions to at least one nucleotide sequence of (a) or (b) encodes a polypeptide having peroxidase-like activity; and
(f) a nucleotide sequence complementary to a nucleotide sequence of (a), (b), (c), (d), or (e).

40. A method of breeding resistance to viral, bacterial, or fungal pathogens into plants, said method comprising:
(a) selecting at least one nontransgenic plant that constitutively expresses a nucleic acid molecule comprising a nucleotide sequence encoding an amino acid selected from the group consisting of the amino acid sequences set forth in SEQ ID NO:2, 4, 6, 8, 10, 12, 14, 17, 19, 21, 23, 25, 27, 29, 31, 33, 35, and 37; wherein said nontransgenic plant expresses said nucleic acid molecule in the absence of pathogen or chemical induction;
(b) using said nontransgenic plant in a breeding program; and
(c) selecting pathogen resistant progeny with desired phenotypic traits.

41. The method of claim 40, wherein said nontransgenic plant is maize.

42. A method of selecting, from a population of plants, plants that are resistant to viral, bacterial, or fungal pathogens and that constitutively express a nucleic acid molecule comprising a nucleotide sequence encoding an amino acid selected from the group consisting of the amino acid sequences set forth in SEQ ID NO:2, 4, 6, 8, 10, 12, 14, 17, 19, 21, 23, 25, 27, 29, 31, 33, 35, and 37, said method comprising:
(a) detecting the expression of said nucleic acid molecule, or evaluating the resistance to viral, bacterial, or fungal pathogens; and
(b) selecting plants that are resistant to viral, bacterial, or fungal pathogens.

43. The method of claim 42, wherein said plants are maize.


SEQUENCE LISTING (37 sequences)

SEQ ID NO: 1 - DNA, 831 bp, Zea mays
  CDS (57)...(716)
aattcggcacgaggaataattagttagttgccttacctgatcagtcatcaccatgcatgagctgctgcatgcagggtggcggcccggcgtacaagttgccactgggcaggcgcgacgggctggcgccggcatcgaacgccgccgtcctagcggcgctcccaccgccgacgtccaaggtgccgacgctgctgtccttcctggcgaagatcaacctggacgtgacggacctggtggcgctgtcgggcgggcacacggtgggcatcgcgcactgcggctccttcgacaaccggctgttcccgacgcaggacccgacgctgaacaagttcttcgcggggcagctgtaccggacctgcccgaccaacgcgacggtcaacacgacggccaacgacgtccgcacgcccaacgccttcgacaacaagtactacgtggacctgctcaaccgggagggcctcttcacgtcggaccaggacctgctgaccaacgccaccacgcgccccatcgtcacgcgcttcgccgtcgaccaggacgccttcttcgaccagttcgtctactcctacgtcaagatggggcaggtcaacgtgctcacgggctcccagggacaggtccgcgccaactgctccgcgcgcaacggcgccgctgctggtgacagtgacctgccgtggtcgtccgtcgtcatcgagacagtcgccgacgccgccggtagcctcgtgctctagataataagcaaataagtagtttgaagctttcttcgcatgcatgttgcaacaaataagcagctagtagcgttgggaataaagcagctagtagcgatcaaaaaaaaaaaaaaaaaaa

SEQ ID NO: 2 - protein, 219 aa, Zea mays
MSCCMQGGGPAYKLPLGRRDGLAPASNAAVLAALPPPTSKVPTLLSFLAKINLDVTDLVALSGGHTVGIAHCGSFDNRLFPTQDPTLNKFFAGQLYRTCPTNATVNTTANDVRTPNAFDNKYYVDLLNREGLFTSDQDLLTNATTRPIVTRFAVDQDAFFDQFVYSYVKMGQVNVLTGSQGQVRANCSARNGAAAGDSDLPWSSVVIETVADAAGSLVL

SEQ ID NO: 3 - DNA, 1354 bp, Zea mays
  CDS (67)...(1008)
aattcggcacgagcttaagcaagtagcttcattcaccgagcgtgcaggcacaggcagcagcttgccatggcgtctcccaccttgatgcaatgcctggtcgccgtttccctcctctcctgtgtcgcccacgcacagctctcgcccacgttctatgcgtcctcctgccccaacctgcagagcatcgttcgggcggcgatgacccaggccgtcgcaagtgagcagaggatgggcgcctctctgctcaggctcttcttccacgactgcttcgttcaaggctgcgacggatcgatccttctcgacgccggaggggagaagaccgccgggccgaacctgaactcggtgcgcggctttgaggtcatcgacaccatcaagcggaacgtcgaggccgcgtgccccggcgtcgtgtcgtgcgccgacatcctcgcgcttgccgcgcgcgacggaaccaaccttctcggcgggccgacctggagcgtgccgctcgggcggcgggactcgacgacggccagcgcctcgctcgccaacagcaaccccccgcccccgacggccagcctcggcacgctcatctccctgttcggcaggcagggcctgtcgccgcgcgacatgacggcgctgtcgggcgcgcacaccatcgggcaggcccggtgcaccaccttccgcggccgcatctacggcgacaccgacatcaacgcctccttcgcggcgctgcggcagcagacgtgcccgcggtccggcggcgacggcaacctggcgcccatcgacgtgcagacgccggtgaggttcgacacggcctacttcaccaacctgctgtcgcggcggggcctgttccactcggaccaggagctcttcaacggcgggtcgcaggacgcgctggtgaggcagtacagcgccagcgcctcgctcttcaacgccgacttcgtggcagccatgattaggatgggcaacgttggggtgctcaccggcaccgccggacagatcaggcgcaactgccgggtcgtcaacagctagatacgacgcatcggattcgatcgatatacttgtagctatagctagcttgctcgtcgaccgagcgcacattgatagatcgaccgacatagagctcgcttctgatgaaccccagtacgtgtactctctagtatatatacatagatatagctatagattgaacacgtcgtcaataccagtagaataagtggtgaacgaccacgcaaggagaagagtgatcgaagcagtgtcacttggttaccgaaatgattcatctgacattttcgtattggattttgaacgcaactatatatatatatatatacactgttgacacctttttcggaaaaaaaaaaaaaaaaaaaaaaaa

SEQ ID NO: 4 - protein, 313 aa, Zea mays
MASPTLMQCLVAVSLLSCVAHAQLSPTFYASSCPNLQSIVRAAMTQAVASEQRMGASLLRLFFHDCFVQGCDGSILLDAGGEKTAGPNLNSVRGFEVIDTIKRNVEAACPGVVSCADILALAARDGTNLLGGPTWSVPLGRRDSTTASASLANSNPPPPTASLGTLISLFGRQGLSPRDMTALSGAHTIGQARCTTFRGRIYGDTDINASFAALRQQTCPRSGGDGNLAPIDVQTPVRFDTAYFTNLLSRRGLFHSDQELFNGGSQDALVRQYSASASLFNADFVAAMIRMGNVGVLTGTAGQIRRNCRVVNS

SEQ ID NO: 5 - DNA, 1263 bp, Zea mays
  CDS (29)...(1099)
aaattatatgcaatcgcaagcgagcagaatggcgaggtccagtggtagtagaccagtggccctcgtgctgctggcgctgtgcgccgccgccctctcgtcggccacggtgaccgtgaatgagcccatcgccaatggcctctcctggagcttctacgacgtttcctgcccgtcggtggagggcatcgtgcgctggcacgtcgccgaggccctccgccgcgacatcggcatcgccgcggggctcatccgcatcttcttccacgactgcttcccgcagggctgcgacgcgtccgtcctcctgtctggttccaacagcgagcagatcgaggtacccaaccagacgctgcgtcccgaggcgctcaagctcatcgacgacatccgcgccgccgtccacgccgtctgcgggcccacggtgtcctgcgccgacatcacaacgctcgccaccagggacgccgtcgtcgcgtccggcggccccttcttcgaggttcctctcgggcggcgcgacggtctggcgccggcgtcaagcgacctggtgggcaccctgccggcgcccttcttcgacgtgccgacgctgatcgagtcgttcaagaaccggagcctggacaaggcggacctggtggcgctgtccggcgcgcacacggtgggccgcggccactgcgtttccttcagcgaccggctgccgcccaacgcggacgatggcaccatggacccggcgttccggcagaggctgacggccaagtgcgccagcgaccccagcgggaacgtggtgacccaggtgctggacgtgcgcacgcccaacgccttcgacaacaagtactacttcgaccttatcgccaagcaggggctcttcaagtcggaccagggcctcatcaaccacccggacaccaagcgcgcggccacccgcttcgcgctcaaccaggccgccttcttcgaccagttcgccaggtccatggtgaagatgagccagatggacatcctcaccggcagcgcgggagagatccgccgcaactgctccgtgcgcaacaccgccctcggagatgtctcatcgtcagcccagctcgagaccaccgcgggcgacgaggggctcgcggccgacgcgtgaaatgatgattagctgctgttgtttttctctggtctccttctgttcaaataaggcttgctgcatcattggtatagtttgcattcttctacccatcaatatggtaaaaaaaaaaaaaaaaaaaaaaaaaaaaaaaaaaaaaaaaaaaaaaaaaaaaaaaaaaaaaa

SEQ ID NO: 6 - protein, 356 aa, Zea mays
MARSSGSRPVALVLLALCAAALSSATVTVNEPIANGLSWSFYDVSCPSVEGIVRWHVAEALRRDIGIAAGLIRIFFHDCFPQGCDASVLLSGSNSEQIEVPNQTLRPEALKLIDDIRAAVHAVCGPTVSCADITTLATRDAVVASGGPFFEVPLGRRDGLAPASSDLVGTLPAPFFDVPTLIESFKNRSLDKADLVALSGAHTVGRGHCVSFSDRLPPNADDGTMDPAFRQRLTAKCASDPSGNVVTQVLDVRTPNAFDNKYYFDLIAKQGLFKSDQGLINHPDTKRAATRFALNQAAFFDQFARSMVKMSQMDILTGSAGEIRRNCSVRNTALGDVSSSAQLETTAGDEGLAADA

SEQ ID NO: 7 - DNA, 1519 bp, Zea mays
  CDS (146)...(1222)
ctcatgtgcgcagcgcgaggctcgagccgccaaagcagggaagcagagagcaagctaataagcaaccgcgtttcccagattccttggcactgcagcagctgcgtccaagcttgctaggtggtggcggcagcagcagcctgcgcctatgtacactgcaatggcagcgcgaccgcttcttcttccccctccggtcctcctcctcctcctcctggtggcggtgctggctgcttcttcggccgcccatggctatggctacggctacggcggcgacgctgctgctgagctcagggtcgggttctacaaggactcgtgcccggacgccgaggccgtcgtccgcaggatcgtcgccaaggccgtccgcgaggaccccacggccaacgcgccgctgctcaggctccacttccacgactgcttcgtccggggctgcgacggctccgtgctcgtcaactccaccagggggaacacggcggagaaggacgccaagcccaaccacacgctggacgccttcgacgtcatcgacgacatcaaggaggcgctggagaagcgctgcccggggaccgtctcctgcgccgacatcctcgccatcgccgccagggacgccgtatcgctggccaccaaggtggtcaccaagggcggctggagcagggacggcaacctctaccaggtggagaccggcaggcgggacggccgcgtgtccagagccaaggaggccgtcaagaacttgccggactccatggatggcatccgcaagctcatcaggaggttcgcttccaagaacctcagcgtcaaggatctcgctgttctctcaggcgcccacgcgatcggcaaatcgcactgcccgtcgatcgccaagcggctgcgcaacttcacggcgcaccgggacagcgacccgaccctggacggcgcgtacgcggcggagctgaggcggcagtgccggagccgcagggacaacacgacggagctggagatggtgccggggagctccaccgcgttcggcacggcctactacggcctggtcgcggagcggagggcgctcttccactccgacgaggcgctgctcaggaacggggagaccagggcgctcgtctaccgctacagggacgcgccgtcggaggcgccgttcctcgcggacttcggggcgtccatgctcaacatgggcagggtgggcgtgctcaccggcgcccagggggagatcaggaagaggtgcgcctttgtcaactagctagcgatgctgtattgtactttgtaccctctcgccttaattaaaatttaaatgctggagtttcaccccggtctcgagagaatcttccatttttactactacatagatttaggaacgtttctaggttttatatgattgtgaaacgtttgggctgagctcatctgtaactgtaaactctgatggaatgcgataacgtgttccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - protein, 358 aa, Zea mays
MYTAMAARPLLLPPPVLLLLLLVAVLAASSAAHGYGYGYGGDAAAELRVGFYKDSCPDAEAVVRRIVAKAVREDPTANAPLLRLHFHDCFVRGCDGSVLVNSTRGNTAEKDAKPNHTLDAFDVIDDIKEALEKRCPGTVSCADILAIAARDAVSLATKVVTKGGWSRDGNLYQVETGRRDGRVSRAKEAVKNLPDSMDGIRKLIRRFASKNLSVKDLAVLSGAHAIGKSHCPSIAKRLRNFTAHRDSDPTLDGAYAAELRRQCRSRRDNTTELEMVPGSSTAFGTAYYGLVAERRALFHSDEALLRNGETRALVYRYRDAPSEAPFLADFGASMLNMGRVGVLTGAQGEIRKRCAFVN

SEQ ID NO: 9 - DNA, 1480 bp, Zea mays
  CDS (154)...(1194)
agtcactcgagcccgcccgggtctcttcctcttccattgattccattcaagatcactactgttaccagccagccagtcgttcgttaagaagagagcgagcgagcaagagagagagagagggagaagggggaagatcagaacgaagagagggagatgagggggcaaacgcagacagcttccgcggggggccggtggcgaggcgacggcgctgcggcgtggtgttggtggtgggtggccgtggtcctcctgcttggccatttaccgagctgcgcgcgcgcggggctgctggagtccaacccgggcctggcctacaacttctacaagaacagctgccccagcgtggactccatcgtccgcagcgtcacctgggcgcaggtcgccgccaaccctgccctcccggctcgcctcctccgcctccacttccatgactgcttcgtcaagggctgcgacgcgtcgatcctgctggacaccgcgcagagcgagaagacggcggcgccgaacctgtcggtgggcgggtacgaggtgatcgacgcgatcaaggcgcagctggagagggcgtgcccgggcgtggtgtcgtgcgccgacatcgtggcgctggcggcgcgcgacgccgtgtcgtaccagttcaaggcgtcgctgtggcaggtggagacggggcggcgcgacggcacggtgtcgctggcgtccaacacgggggcgctgccgtcgcccttcgcgggcttcgcgggcctgctgcagagcttctcggaccgggggctgaacctgacggacctcgtggcgctgtcgggggcgcacaccatcggcgtggcaagctgctccagcgtcaccccgcgcctgtaccaggggaacgccagcagcgtggacccgctgctggactcggcctacgcgcggacgctcatgtcgtcgtgccccaacccgtcgccggcgtcggccaccgtggcgctggacggcggcacgccgttccggttcgacagcagttactactccagggtgcagcagaagcagggcacgctggcctccgacgccgcgctggcgcagaacgccgccgccgcgcagatggtggccgacctcaccaaccccatcaagttctacgccgccttctccatgtccatgaagaagatgggccgcgtcgacgtgctcaccggcgccaacggccagatcaggaagcagtgccgccaggtcaacacctcctgatcaacaaggcctcacagggaggggccatggtttttccctcttcgttcttctctttttctgcaccgcctggtattattgttccatgcatccggtttcggctcggcttcggctcggcgcgcatcaaatatatttcgcccccgcgccgtgattgagggccctcctccgacgaaagaaatccattgctttctttgttatttatattagtttactcactgtcctgtgttccattgtcttgccttgcaaaaatgtaagaaagattaaagatgtaagcaaaaaaaaaaaaaaa

SEQ ID NO: 10 - protein, 346 aa, Zea mays
MRGQTQTASAGGRWRGDGAAAWCWWWVAVVLLLGHLPSCARAGLLESNPGLAYNFYKNSCPSVDSIVRSVTWAQVAANPALPARLLRLHFHDCFVKGCDASILLDTAQSEKTAAPNLSVGGYEVIDAIKAQLERACPGVVSCADIVALAARDAVSYQFKASLWQVETGRRDGTVSLASNTGALPSPFAGFAGLLQSFSDRGLNLTDLVALSGAHTIGVASCSSVTPRLYQGNASSVDPLLDSAYARTLMSSCPNPSPASATVALDGGTPFRFDSSYYSRVQQKQGTLASDAALAQNAAAAQMVADLTNPIKFYAAFSMSMKKMGRVDVLTGANGQIRKQCRQVNTS

SEQ ID NO: 11 - DNA, 1183 bp, Zea mays
  CDS (60)...(1079)
ataacatcaccatgacttgaactaagcgatacagcaaacttgttgagactcagctgatcatgggtcgatacatgttggcgcccgtgttggcagcactcgtcgtcgccgcctcctcatcggtggcatcacacgcctcgccgccggggaaacttgaggtcggtttctacgagcactcgtgcccacaggccgaggacatcgtgcgcaacgccgtccgccgcggcatagcccgcgagcccggcgtcggcgcggggctcatccgcatgcacttccacgactgcttcgtccggggctgcgacggctccatcctcatcaactccacgccggacaacaaggccgagaaggactccgtggccaacaaccccagcatgcgtggcttcgacgtcgtcgacgacgccaaggccgtcctcgaggcgcactgcccgcgcaccgtctcctgcgccgacatcgtcgcgttcgcagcccgtgacagcgcctacctcgccggcggcctggactacaaggtcccgtctggccgccgtgacggccgcgtgtccaaagaggatgaggtgctcgacaacaatgtccctgccccgactgacgaggtcgacgagctcatcgagagcttcaagcgcaaggggctcaacgccgacgacatggtcacgctctctggcgcgcacaccatcgggcgctcccactgctcctcgttcacggagcgcctctacaacttcagcgggcagctggggcggacggacccgtccctcgaccctgcctacgctgagcacctcaagatgcgctgcccatggccgtccagcaacgaccagatggaccccacggtggtgccgcttgacccagtcacgccggcgaccttcgacaaccagtactacaagaacgtgctggcgcacaaggtcctgttcatctccgacaacacactgctcgaaaacccatggactgccggaatggtccacttcaacgccgcagtcgagaaggcatggcaggtcaagttcgccaaggccatggttaagatgggcaaggtccaggtgctcaccggcgacgagggagagatcagggagaagtgcttcgccgtcaacccacactactagtcgtcaagtttagctaattaccccgcgaattatgtttgtttatacgttccatacatcaacattatgtagagtgtttttcgttctaaaaaaaaaaaaaaaaaaaa

SEQ ID NO: 12 - protein, 339 aa, Zea mays
MGRYMLAPVLAALVVAASSSVASHASPPGKLEVGFYEHSCPQAEDIVRNAVRRGIAREPGVGAGLIRMHFHDCFVRGCDGSILINSTPDNKAEKDSVANNPSMRGFDVVDDAKAVLEAHCPRTVSCADIVAFAARDSAYLAGGLDYKVPSGRRDGRVSKEDEVLDNNVPAPTDEVDELIESFKRKGLNADDMVTLSGAHTIGRSHCSSFTERLYNFSGQLGRTDPSLDPAYAEHLKMRCPWPSSNDQMDPTVVPLDPVTPATFDNQYYKNVLAHKVLFISDNTLLENPWTAGMVHFNAAVEKAWQVKFAKAMVKMGKVQVLTGDEGEIREKCFAVNPHY

SEQ ID NO: 13 - DNA, 1407 bp, Zea mays
  CDS (142)...(1185)
cgcgactagtacactgcctgccaaacagctgagcagagtagagtagagcagagggcgaagtgcagcgcagtgtgccccgtctcggaacagcgcggaccaccagagcgtgcgcgtccgtgccccacccctccctctctatccatggcggtcccccgcgggtgcctcgggctccccctggtcgccgtgctcctcgcgtccctctgccgcggccaggcggcggtgagggagctcaaggtcgggtactacgccgagacgtgcccggaggccgaggacatcgtccgtgagaccatggcgcgcgcgcgcgcacgcgaggcccgcagcgtcgcctccgtcatgcgcctccagttccacgactgcttcgtcaacgggtgcgacggctcggtgctgatggacgccacgccgacgatgcccggcgagaaggatgcgctctccaacatcaactccctgcgctcgttcgaggtcgtcgacgagatcaaggacgcgctggaggagcgctgccccggagtggtctcctgcgccgacatcgtcatcatggccgcccgcgacgccgtcgtcctgaccggtgggcctaactgggaggtgcggctcgggcgcgaggacagcatgacggcgagccaggaggacgcggacaacatcatgccgagcccgcgcgcaaacgcgagcgctctcatccggctcttcgcggggctcaacctcagcgtcaccgacctggtcgcgctctcgggctcgcactccatcggcgaggcccgctgcttctccatcgtcttccgcctctacaaccagtctggatccggccgccccgacccgcacatggacaccgcctaccgccgctcgctcgacgcactctgccccaagggcggcgacgaggaggtcacgggaggcctagacgccaccccacgcgtcttcgacaaccagtacttcgaggacctcgtcgcgctccgcggcttcctcaactccgaccagacgctcttctctgacaacaccaggacccgtcgcgtcgtcgagcggctcagcaaggaccaggacgccttcttcagggccttcatcgaggggatgataaagatgggggagctccaaaaccccaggaaaggggagatacggcgcaactgtcgcgttgctaacaactcgccgtggcaaccaaggacggggatggcgtccggacagtcgacatctgagctccggtgatgaggttggtgtttcagaagaaatcgagccctgatatggtactaatatgttgacatgcattgttgtttttttggtcgtgtgtaagttttgcacctacctatggctgtggtgcccgagctgcgctcattgctgacgtggggaataattgagacattgtgccttagctccaataacgttcaatatatttatcctttaaaaaaaaaaaaaaaaaaaaaaaaaaaa

SEQ ID NO: 14 - protein, 347 aa, Zea mays
MAVPRGCLGLPLVAVLLASLCRGQAAVRELKVGYYAETCPEAEDIVRETMARARAREARSVASVMRLQFHDCFVNGCDGSVLMDATPTMPGEKDALSNINSLRSFEVVDEIKDALEERCPGVVSCADIVIMAARDAVVLTGGPNWEVRLGREDSMTASQEDADNIMPSPRANASALIRLFAGLNLSVTDLVALSGSHSIGEARCFSIVFRLYNQSGSGRPDPHMDTAYRRSLDALCPKGGDEEVTGGLDATPRVFDNQYFEDLVALRGFLNSDQTLFSDNTRTRRVVERLSKDQDAFFRAFIEGMIKMGELQNPRKGEIRRNCRVANNSPWQPRTGMASGQSTSELR

SEQ ID NO: 15 - DNA, 1565 bp, Zea mays
  intron (315)...(491)
gcacaataatacagcaaaggaggctagcagaagtgcaggattaataagctaagctagtagaaattaagcaaagcataggcacagccatggctacctcctctggttcttgccttattattagcctgttggtggtggtggtggcggcggcgctgtcggcctcaacggcgtcggcacagctgtcgtcgacgttctacgacacgtcgtgccccagcgcgatgtccaccatcagcagcggcgtgaactccgccgtggcgcagcaggctcgtgtgggggcgtcgctgctccggctccacttccacgactgcttcgtccaagcaagtctagctgtctcagatgcatctatctatctacttatatataagcatgatttcctttctagctagctagcatcgtcgtgcattttaatttgaagataaaagattagcacgtcgtatatgcatgcgattaattaaccaggaggcatcatggtgaaatttctggtggtccaccagggctgcgacgcgtccattctgctgaacgacacgtccggggagcagacccagccgccgaacctaactctgaacccgagggccttcgacgtcgtcaacagcatcaaggcgcaggtggaggcggcgtgcccgggcgtcgtctcctgcgccgacatcctcgccgtcgccgcccgcgacggagttgtcgcgctcggcgggccttcgtggaccgtgcttctgggcagaagggactcgaccggttcgttccctagccagacaagcgacctcccacctccgacgtcgagcctccaagcactgttagccgcgtacagcaagaagaacctcgacgcgaccgacatggtcgctctctcaggcgctcacacaatcgggcaggcccagtgctcgagcttcaacggccacatctacaacgacacgaacatcaacgcggccttcgcgacgtcgctcaaggccaactgccccatgtccggcggcagcagcctggcgccgctggacaccatgaccccgaccgtgttcgacaacgactactacaagaacctgctgtcgcagaaggggctgctgcactcggaccaggagctgttcaacaacggcagcaccgacagcacggtcagcaactttgcgtccagcttcggccgccttcaccagcgccttcacggcggccatggtgaagatggggaacctcggcccgctcaccgggaccagtgggcagatcaggctcacctgctggaagctcaactcgtcctaataattaaggacggacgtccgatagacgatcctgcgcaatcgtatcgtacgtgcatgatacgcatacatctggaaactactataccaatgcaaacagagatctatacgtacgagtatgtataacgacgagtgatgtttgtatggatctacgtatgtaacaaggacctctcgtagcgcaaaggcgcgcgttgggagattaattaggtacacaagctattaccacattatatatcactctcattgtggctacatatctatatctctgaggccaaatgcttgggtgtccagtactaattaataataattcagtgcgtatgcaaaaaaaaaaaaaaaaaa

SEQ ID NO: 16 - DNA, 1388 bp, Zea mays
  CDS (87)...(1175)
gcacaataatacagcaaaggaggctagcagaagtgcaggattaataagctaagctagtagaaattaagcaaagcataggcacagccatggctacctcctctggttcttgccttattattagcctgttggtggtggtggtggcggcggcgctgtcggcctcaacggcgtcggcacagctgtcgtcgacgttctacgacacgtcgtgccccagcgcgatgtccaccatcagcagcggcgtgaactccgccgtggcgcagcaggctcgtgtgggggcgtcgctgctccggctccacttccacgactgcttcgtccaaggctgcgacgcgtccattctgctgaacgacacgtccggggagcagacccagccgccgaacctaactctgaacccgagggccttcgacgtcgtcaacagcatcaaggcgcaggtggaggcggcgtgcccgggcgtcgtctcctgcgccgacatcctcgccgtcgccgcccgcgacggagttgtcgcgctcggcgggccttcgtggaccgtgcttctgggcagaagggactcgaccggttcgttccctagccagacaagcgacctcccacctccgacgtcgagcctccaagcactgttagccgcgtacagcaagaagaacctcgacgcgaccgacatggtcgctctctcaggcgctcacacaatcgggcaggcccagtgctcgagcttcaacggccacatctacaacgacacgaacatcaacgcggccttcgcgacgtcgctcaaggccaactgccccatgtccggcggcagcagcctggcgccgctggacaccatgaccccgaccgtgttcgacaacgactactacaagaacctgctgtcgcagaaggggctgctgcactcggaccaggagctgttcaacaacggcagcaccgacagcacggtcagcaactttgcgtccagcttcggccgccttcaccagcgccttcacggcggccatggtgaagatggggaacctcggcccgctcaccgggaccagtgggcagatcaggctcacctgctggaagctcaactcgtcctaataattaaggacggacgtccgatagacgatcctgcgcaatcgtatcgtacgtgcatgatacgcatacatctggaaactactataccaatgcaaacagagatctatacgtacgagtatgtataacgacgagtgatgtttgtatggatctacgtatgtaacaaggacctctcgtagcgcaaaggcgcgcgttgggagattaattaggtacacaagctattaccacattatatatcactctcattgtggctacatatctatatctctgaggccaaatgcttgggtgtccagtactaattaataataattcagtgcgtatgcaaaaaaaaaaaaaaaaaa

SEQ ID NO: 17 - protein, 362 aa, Zea mays
MATSSGSCLIISLLVVVVAAALSASTASAQLSSTFYDTSCPSAMSTISSGVNSAVAQQARVGASLLRLHFHDCFVQGCDASILLNDTSGEQTQPPNLTLNPRAFDVVNSIKAQVEAACPGVVSCADILAVAARDGVVALGGPSWTVLLGRRDSTGSFPSQTSDLPPPTSSLQALLAAYSKKNLDATDMVALSGAHTIGQAQCSSFNGHIYNDTNINAAFATSLKANCPMSGGSSLAPLDTMTPTVFDNDYYKNLLSQKGLLHSDQELFNNGSTDSTVSNFASSFGRLHQRLHGGHGEDGEPRPAHRDQWADQAHLLEAQLVLIIKDGRPIDDPAQSYRTCMIRIHLETTIPMQTEIYTYEYV

SEQ ID NO: 18 - DNA, 1467 bp, Zea mays
  CDS (109)...(1095)
gcgacaggacgagactcgcagctagctgacacggccgagaagcagcttgcattgcaggcgtagtacgtacccagcagcagctagcactagcagtccatcggagcgacgatggtgagaaggacggtgctggcggcgctgctggtggccgccgccctcgccggcggcgcgcgggcgcagctcaaggaggggttctacgactactcctgcccacaggcggagaagatcgtcaaggactacgtgaaggcgcacatcccccacgcgcccgacgtcgcctccaccctgctccgcacccacttccacgactgcttcgtcaggggctgcgacgcgtcagtgctgctcaacgcgacgggcggcagcgaggcggagaaggacgcggcgcccaacctgacgctgcgcggcttcggcttcatcgaccgcatcaaggcgctgctcgagaaggagtgccccggcgtggtgtcctgcgccgacatcgtcgcgctcgccgcccgcgactccgtcggcgtcatcggcggtccgttctggagcgtgccgacggggaggcgcgacggcaccgtgtccatcaagcaggaggcgctggaccagatccccgcgcccaccatgaacttcacccaactcctccagtccttccagaacaagagcctcaacctcgccgacctcgtctggctctcaggggctcacacgatcggcatctcccaatgcaactccttcagcgagcgcctgtacaacttcacggggcgcggcgggcccgacgacgcggacccgtcgctggacccgctgtacgccgcgaagttgcggctcaagtgcaagacgctgacggacaacacgacgatcgtggagatggaccccggcagcttccgcaccttcgacctgagctactaccgcggcgtgctcaagcggcggggcctgttccagtccgacgccgcgctcatcaccgacgccgcctccaaggccgacatcctcagcgtgatcaacgcgccgcccgaggtgttcttccaggtcttcgcgggctccatggtcaagatgggcgccatcgaggtcaagaccggctccgagggcgagatcaggaagcactgcgccctcgtcaacaagcactaggcggcggaattcatgcgggagatggctccattgctcgcaaaaaaatccttgtgagacacacaacatgctctgcatctgcaggcgttgtcgtcaccttggtggacgtagtacatcggtgcatggattatctgttgtttaatttgtacgttcagttcattctgtttcttgtattcttttggttcctttcctcttgtttattcatggatgatgaggtgtttctgttttattctctggttcagctgtaaccatgtaacatgtaaggtgcgcgttttgtcctcgtgatctctgcaactgtatttattttaatggatggtatacatggagatatgatcatgataataacaataaggtgattacaaaaaaaaaaaaaaa

SEQ ID NO: 19 - protein, 328 aa, Zea mays
MVRRTVLAALLVAAALAGGARAQLKEGFYDYSCPQAEKIVKDYVKAHIPHAPDVASTLLRTHFHDCFVRGCDASVLLNATGGSEAEKDAAPNLTLRGFGFIDRIKALLEKECPGVVSCADIVALAARDSVGVIGGPFWSVPTGRRDGTVSIKQEALDQIPAPTMNFTQLLQSFQNKSLNLADLVWLSGAHTIGISQCNSFSERLYNFTGRGGPDDADPSLDPLYAAKLRLKCKTLTDNTTIVEMDPGSFRTFDLSYYRGVLKRRGLFQSDAALITDAASKADILSVINAPPEVFFQVFAGSMVKMGAIEVKTGSEGEIRKHCALVNKH

SEQ ID NO: 20 - DNA, 1522 bp, Zea mays
  CDS (187)...(1170)
ggctagctagctatctagagagctcatcatatcgctgctcgctctcatccaccattatagagaagagcagatcgagctgcagctggcagaggccgagttgttgctagctagctcctgcttgctaaatttgcatcgtatccgatccattccatgaagaagtcgtcgatgatggcgcccatgacgatcatggcgagagttgccgctgtgctcgtcctctcgtcggctgccatggcttccgccgcaggagcagctgggctggacatgaatttctacggcagcacgtgcccgcgcgtggaggccatcgtcaaggaggagatggtggcgaccctcaaggcggcgccgacgctggccggcccgctgctccgcctccatttccacgactgcttcgtcaggggctgcgacgcctccgtgctcctggactcgactcccaccagcacggcggagaaggacgccaccccgaacctcaccctccggggcttcggctccgtgcagcgcgtcaaggaccggctggaggaagcgtgcccgggcaacgtctcctgcgccgacgtcctggcgctcatggcgcgcgacgccgtcgtgctggccaacgggccctcctggcccgtcgcgctgggccgccgctacggccgcgtctccctcgccaacgagaccaaccagctgcccccgcccaccgccaacttcacccgcctcgtcagcatgttcgccgccaagggcctctccgtcagggacctcgtcgtgctctccggcggccacaccctcggcaccgcgcactgcaacctcttcagcgaccgcctctacaacttcaccggcgccaacagcctcgccgacgtcgacccggcgctcgacgccgcctacctcgcccgcctcaggtccaggtgccggagcctcgccgacaacaccacgctcaacgagatggaccccggcagcttcctcagcttcgactccagctactacagcctggtggccaggcgccgggggctcttccactccgacgccgcgctgctcaccgacccggccaccagggcgtacgtccagcgccaggccacggggctcttcaccgccgagttcttccgcgacttcgccgactccatggtcaagatgtccaccatcgacgtgctcaccggccagcagcagggcgagatcagaaagaaatgcaacctcgtcaactgacaaataatacgtacattaattgctgctggttttgacgatgcctttcacttacgttcatccacttaattcatgcatccatgttggttgggttcattcaattattatattcgttgcttacctttacttttgcttggttaatcttaattaaatcgtgtgagttttcttttcttttcttgagtatatatatgtatgtacgtagagcgtgatgagtgccttttcaagtttattgtttttttactttttcctctcgtgcatatggttcaaatccatgatgatgatgtaatgcgcatgtaattaataatgacaatcaagtcaataaacacacatgcaatttaaaaaaaaaaaaaaaaaa

SEQ ID NO: 21 - protein, 327 aa, Zea mays
MARVAAVLVLSSAAMASAAGAAGLDMNFYGSTCPRVEAIVKEEMVATLKAAPTLAGPLLRLHFHDCFVRGCDASVLLDSTPTSTAEKDATPNLTLRGFGSVQRVKDRLEEACPGNVSCADVLALMARDAVVLANGPSWPVALGRRYGRVSLANETNQLPPPTANFTRLVSMFAAKGLSVRDLVVLSGGHTLGTAHCNLFSDRLYNFTGANSLADVDPALDAAYLARLRSRCRSLADNTTLNEMDPGSFLSFDSSYYSLVARRRGLFHSDAALLTDPATRAYVQRQATGLFTAEFFRDFADSMVKMSTIDVLTGQQQGEIRKKCNLVN

SEQ ID NO: 22 - DNA, 1451 bp, Zea mays
  CDS (170)...(1198)
ggaaacagctattgaccatgattacgcccaagttctatacgactcactataggaaagcttgtacgcctgcaggtaccggtcccggaattcccgggtcgacccacgcgtccgcacctgatattttttccgacggacacacaagcatatatacatacacgccgacatagggatgatggcttcctctagtcctcagcgtgcagccggcgccgccgtcgccggcgtgctgctggtggcggctgccgccctgtgctcgtgcggcgccatggcgcagctgaccgcggactactacgactgcacatgccctgacgcctacaacatcgtgaagcaggtcctgatcgaggcgcacaagtccgacgtgcgcatctacgccagcctcacctgcctccacttccacgactgcttcgtccagggctgcgatggctcggtgctcctggacgccgttccgggggtggccaactccactgagaagctggcgccggccaacaacaactcggcgcgagggttcccggtggtggacaaagtgaaggcggcgctggaggacgcctgccccggcgtcgtctcctgcgccgacatcctcgccctcgccgcagagatctcggtcgaactgtccggaggaccaaagtgggcagtgcttcttgggaggctagacagcaagaaggccgacttcaagagcgcggagaacctgccgtcgccgttcgacaacctgacggtgctggagcagaagttcgcggccgtgggcctccacaccgtggacctggtggccctctcgggagctcacacgttcgggcgggtccagtgccagttcgtcacggggcggctgtacaacttcagcggcacgaaccggccggaccccacgctcaactccggctaccgggcgttcctggcccagaggtgcccccagaacggcagcccctcggccctcaacgacctggaccccacgacgccgaacctgttcgacaaccactactacaccaacctggaggtgaaccggggcttcctcggctcggaccaggagctcaagtcggcgccgcaggcgcagggcgtcaccgcgcccgtcgtcgaccagttcgccaccagccaggccgccttcttcagcagcttcgcgcagtccatgatcaacatgggcaacatccagccgctcaccgacccggccaagggagaggtccgctgcgactgccgggtggctaatgatgattaatccacgacgacgacgtgtgtagttagaaacggacgtgaatgcatgtgcatgtgcccggccgggtcgttagtacaccgacaagcaagagtgcactagcgctatggatcggactatatggtgcacaactgcacatgcatgcatataatatcagggcctcgtcgtttcgaattgtttgctgtcgacttcaattaatagtttctcataaatgtgcaataaagaggaacccaatttcttcattaaaaaaaaaaaaaaa

SEQ ID NO: 23 - protein, 342 aa, Zea mays
MMASSSPQRAAGAAVAGVLLVAAAALCSCGAMAQLTADYYDCTCPDAYNIVKQVLIEAHKSDVRIYASLTCLHFHDCFVQGCDGSVLLDAVPGVANSTEKLAPANNNSARGFPVVDKVKAALEDACPGVVSCADILALAAEISVELSGGPKWAVLLGRLDSKKADFKSAENLPSPFDNLTVLEQKFAAVGLHTVDLVALSGAHTFGRVQCQFVTGRLYNFSGTNRPDPTLNSGYRAFLAQRCPQNGSPSALNDLDPTTPNLFDNHYYTNLEVNRGFLGSDQELKSAPQAQGVTAPVVDQFATSQAAFFSSFAQSMINMGNIQPLTDPAKGEVRCDCRVANDD

SEQ ID NO: 24 - DNA, 1334 bp, Zea mays
  CDS (62)...(1075)
actttgttgctgtttttattcgtttgtcacataagcagtggtggtcggtcggccggcgacaatgagcggccgtgtgttcgtcctggccgctgccttcggcgtgctgctcgccgcagccgccgtgtcctcgaggcccgtcctgactccgctgggtactagccgccctgctgccggcgacctgtccgtgtacttccacgcggactcgtgcccgcagctggagacgatcgtgcggtccagcgtggacgcggcgctccagcagaacgtccgtctgacggcgggtctcctccgcctcttgttccacgactgcttcccgcagggctgcgacgcgtccatcctcctggacaacggcgagcgcggcctcccgcccaacgtggggctgcagcaggaggccgtgcagctggtggaggacatccgcgggaaggtgcacgcggcgtgcgggcccaccgtgtcgtgcgccgacatcacggtgctggccacccgcgacgccgtgagcctgtccggcgggccttccttcacggtgccgctggggcggctggacagcgcggcgccggcgtccagcaacgacgtgttcacgctgccgccgccgacggcgacggtggacgagctgctgacggcgttcgggagcaagaacctgtcggacccggcggacctggtggcgctgtcgggcgcgcacacggtggggaaggcgcggtgcagctcgtttggcgacgtggcgggcccggccaccgacgacgtgacgcggtgcgtgacggcgacgtgctcggcccccgggagcggcgacacgctgcgggacctggacttcctgacgcccgccgtgttcgacaacctctacttcgtggagctgacgctgaggaagaacaagggggtgatgctgccgtcggaccaggggctggtgagcgacccgcgcacgagctggctcgtccagggcttcgccgacaaccactggtggttcttcgaccagttcaggacctccatgatcaagatgagccagctcaggggaccccaggggaacgtcggcgagatccgccgtaactgcttccgcccaaacaccaacggcatcgccgcctctgcttgagttgactgaccaggcaccgatccattacttcactttcactgtgtgtaataattaataataataaaacaaaaacaacgccagcccgtacgtgccgtgtgaggggggcacgtggttgtggctatactgtagccgtcaccaccatgtcggcgtcgtccatgcatgtcgcattaccgatttttgttcatgttccgatctcatctcatcatctcgctatcaataacaattataatagtgtttattagtaaaaaaaaaaaaaaaa

SEQ ID NO: 25 - protein, 337 aa, Zea mays
MSGRVFVLAAAFGVLLAAAAVSSRPVLTPLGTSRPAAGDLSVYFHADSCPQLETIVRSSVDAALQQNVRLTAGLLRLLFHDCFPQGCDASILLDNGERGLPPNVGLQQEAVQLVEDIRGKVHAACGPTVSCADITVLATRDAVSLSGGPSFTVPLGRLDSAAPASSNDVFTLPPPTATVDELLTAFGSKNLSDPADLVALSGAHTVGKARCSSFGDVAGPATDDVTRCVTATCSAPGSGDTLRDLDFLTPAVFDNLYFVELTLRKNKGVMLPSDQGLVSDPRTSWLVQGFADNHWWFFDQFRTSMIKMSQLRGPQGNVGEIRRNCFRPNTNGIAASA

SEQ ID NO: 26 - DNA, 1285 bp, Zea mays
  CDS (96)...(1058)
gctcgatcggcgatctgtagtagcagcactagcactagctacgtagtacattgcatcgtcgtctctttgctagtagtagctatctcctggccgccatggcgtcttcctcggtctcatcctgcctgctgcttctcctgtgcttggcggcggtggcgtccgcgcagctctcgccgacgttctacgactcgtcgtgccccaacgcgctgtccaccatcaagagcgccgtgaacgccgccgtgcagaaggagaaccgcatgggggcctccttgctcaggctgcacttccatgactgctttgtccaggggtgcgacgcgtcggtgctgcttgccgacaacgccgccacgggcttcaccggcgagcagggtgctgcgcccaacgccgggtcgctgaggggcttcgacgtcatcgccaacatcaaggcacaggtggaggcagtctgcaagcagaccgtctcctgcgccgacatcctcgccgtcgccgcccgtgattccgtcgtcgcgttgggcgggccgtcatggacggttcctctggggcggagggactcgacgacggcgagcctgtccctggcgaacagcgacctgccgcctcccttcttcaacctcggccagctcataacagcgttcggcaacaagggtttcaccgcgaccgagatggccacgctctccggcgcgcacaccatcgggcaggcgcagtgcaagaacttcagggaccacatctacaacgacaccaacatcaaccagggcttcgcgagctcgctcaaggccaactgcccccggcccaccggctccggcgacggcaacctggcgccgctcgacaccaccacgccgtacagcttcgacaacgcctactacagcaacctgctgagccagaaggggctcctgcactcggaccaggagctcttcaacggcggcagcaccgacaacaccgtcaggaacttcgcgtccaactcggccgccttcagcagcgccttcgccgcggccatggtgaagatgggcaacctcagcccgctcaccggatctcagggccagatcaggcttacctgctccacagtgaactaagattaataataatcaccatacgcaaaaaagagtggtatattcacatgcgaataataaggctaagccaccatgtgactagctagtggtatattcaacgaatcgtttgaaagggacgagtgcgactttaattttagatactagctgcaaaagcggcatatatgtatcagcccgatcaattgctcaaagaaaagatgcacgtcatacatttcaaaaaaaaaaaaaaaaaa

SEQ ID NO: 27 - protein, 320 aa, Zea mays
MASSSVSSCLLLLLCLAAVASAQLSPTFYDSSCPNALSTIKSAVNAAVQKENRMGASLLRLHFHDCFVQGCDASVLLADNAATGFTGEQGAAPNAGSLRGFDVIANIKAQVEAVCKQTVSCADILAVAARDSVVALGGPSWTVPLGRRDSTTASLSLANSDLPPPFFNLGQLITAFGNKGFTATEMATLSGAHTIGQAQCKNFRDHIYNDTNINQGFASSLKANCPRPTGSGDGNLAPLDTTTPYSFDNAYYSNLLSQKGLLHSDQELFNGGSTDNTVRNFASNSAAFSSAFAAAMVKMGNLSPLTGSQGQIRLTCSTVN

SEQ ID NO: 28 - DNA, 1159 bp, Zea mays
  CDS (7)...(969)
gtggcgatggcagtactgcacttgcaggcggcggcggtggcggtgctgctgatggcgacggggctgcgcgcgcagctgcgtgtgggcttctacgacagctcgtgcccagcggcggagatcatcgtgcagcaggaggtgagcagggcggtggcggccaacccgggcctcgccgccggcctgctccgcctccacttccacgactgtttcgttgggggctgcgacgcgtccgtgctcatcgactccaccaagggcaacaccgcggagaaggacgccgggcccaacctcagcctgcggggcttcgaggtcgtcgatcgcatcaaggcgcgcgtcgagcaggcctgcttcggcgtcgtctcctgcgcggacatactcgcgttcgcggccagggacagcgtcgcactcgccggcgggaacgcgtaccaggtgccggcggggcggcgggacgggtccgtgtcgcgcgcgtcggacaccaacggcaacctgccgccgccgacggccaacgtcgcgcagctcacgcagatcttcggcaccaagggcctgacgcagaaggagatggtcatcctgtcgggcgcgcacaccatcggctcctcgcactgcagctccttcagcggccggctgtcggggtcggccacgacggcgggcgggcaggacccgaccatggacccggcgtacgtggcgcagctggcgcggcagtgcccgcagggcggcgacccgctcgtgcccatggactacgtctcccccaacgccttcgacgagggcttctacaagggcgtcatgtccaaccgcggcctgctgtcctcggaccaggcgctgctcagcgacaagaacaccgccgtgcaggtcgtcacctacgccaacgacccggccaccttccaggccgacttcgccgccgccatggtcaagatgggctccgtcggcgtgctcaccggcaccagcggcaaggtcagggccaactgcagagtcgcctgatccatcatcatcattcactcgtgtggagttgtagattcgttatattgattgatttggaccggacgacgtcgacgggatggcgtagcatagcatagtctgcttgcgcaattgtatgtattgcctgtacttgcgcgtgtatgggtttcgctgtgcaaattttcgttggctccccaaaaaaaaaaaaaaaaaa

SEQ ID NO: 29 - protein, 320 aa, Zea mays
MAVLHLQAAAVAVLLMATGLRAQLRVGFYDSSCPAAEIIVQQEVSRAVAANPGLAAGLLRLHFHDCFVGGCDASVLIDSTKGNTAEKDAGPNLSLRGFEVVDRIKARVEQACFGVVSCADILAFAARDSVALAGGNAYQVPAGRRDGSVSRASDTNGNLPPPTANVAQLTQIFGTKGLTQKEMVILSGAHTIGSSHCSSFSGRLSGSATTAGGQDPTMDPAYVAQLARQCPQGGDPLVPMDYVSPNAFDEGFYKGVMSNRGLLSSDQALLSDKNTAVQVVTYANDPATFQADFAAAMVKMGSVGVLTGTSGKVRANCRVA

SEQ ID NO: 30 - DNA, 1310 bp, Zea mays
  CDS (100)...(1098)
cgcacgctagtgtcagtggcacgcagggcagccgcgcgcaccgaggggaggccagagctagcggcgaggccaagggcgcgggcgcgggcgcggacggcaatggcgagagctggaggcggcggtccggtccgcggcgcggcgctggtggtcgtgctgctaggcatcgtcgtgggcgcggcgagggctcagctgcggcagaactactacggcagctcgtgccccagcgcggagtccacggtgcgctccgtcatctcgcagcgcctccagcagagcttcgccgtggggcccggcacgctccgcctcttcttccacgactgcttcgtcaggggatgcgacgcgtcggtgatgctgatggcgccgaacggggacgacgagagccacagcggcgcggacgccacgctgtcgccggacgccgtggacgccatcaacaaggcgaaggcggccgtggaggcgctccccgggtgcgccggcaaggtctcgtgcgcggacatcctcgccatggccgcacgtgacgtcgtctccctgctgggcggtccgagctacggcgtggagctggggcggctggacggcaagacgttcaacagggccatcgtgaagcacgtcctcccgggccccggcttcaacctggaccagctcaacgccctcttcgcgcagaacggcctcacgcagacggacatgatcgcgctctcaggcgcgcacacgatcggggtgacgcactgcgacaagttcgtgcggcggatctacacgttcaagcagcggctggcgtggaacccgccgatgaacctggacttcctgcgctcgctgcggcgggtgtgccccctcagctacagccccacggcgttcgccatgctggacgtcaccacgcccagggtcttcgacaacgcctacttcaacaacctccgctacaacaagggcctgctcgcctccgaccaggtgctcttcaccgaccgccgctcccgacccaccgtcaacctcttcgccgccaacgccaccgccttctacgaggcattcgtcgccgccatggccaagctcggcaggatcggcctcaagaccggcgccgacggcgagatacgccgcgtctgcaccgccgtcaactaagcctgcattggctgcttgctgcttgcgtgcgtgggtttcacttatttcacttcttctttctcttgtttatatacgtacgtttgtcggatggattttggtagccatgagatgacatccttgctctgagctagcggacctgccccgattggatgatatagattgatccagatgttcttctaaaaaaaaaaaaaaaaaaaaaaaaaaaaaaaaaa

SEQ ID NO: 31 - protein, 332 aa, Zea mays
MARAGGGGPVRGAALVVVLLGIVVGAARAQLRQNYYGSSCPSAESTVRSVISQRLQQSFAVGPGTLRLFFHDCFVRGCDASVMLMAPNGDDESHSGADATLSPDAVDAINKAKAAVEALPGCAGKVSCADILAMAARDVVSLLGGPSYGVELGRLDGKTFNRAIVKHVLPGPGFNLDQLNALFAQNGLTQTDMIALSGAHTIGVTHCDKFVRRIYTFKQRLAWNPPMNLDFLRSLRRVCPLSYSPTAFAMLDVTTPRVFDNAYFNNLRYNKGLLASDQVLFTDRRSRPTVNLFAANATAFYEAFVAAMAKLGRIGLKTGADGEIRRVCTAVN

SEQ ID NO: 32 - DNA, 1170 bp, Zea mays
  CDS (25)...(1092)
gggcggcagcagcagcctgcgcctatgtacactgcaatggcagcgcgaccgctgcttcttccccctccggtcctcctcctcctggtggtgctggctgcttcgtcggccgcccatggctacggcgcctacggctacggcgacgctgctgctgagctcagggtcgggttctacaaggactcgtgcccggacgccgaggccgtcgtccgcaggatcgtcgccaaggccgtccaagaggaccccacggccaacgcgccgctgctcaggctccacttccacgactgcttcgtccggggctgcgacggctccgtgctcgtcaactccaccagggggaacacggcggagaaggacgccaagcccaaccacacgctggacgccttcgacgtcatcgacgacatcaaggaggcgctggagaagcgctgcccggggaccgtctcctgcgccgacatcctcgccatcgccgccagggacgccgtctcgctggccaccaaggtggtgaccaagggcggctggagcagggacggcaacctctaccaggtggagaccggcaggcgggacggccgcgtgtccagagccaaggaggccgtcaagaacttgccggactccatggatggcatccgcaagctcatcaggaggttcgcttccaagaacctcagcgtcaaggatctcgctgttctctcaggcgcccacgcgatcggcaaatcgcactgcccgtcgatcgccaagcggctgcgcaacttcacggcgcaccgggacagcgacccgaccctggacggcgcgtacgcggcggagctgaggcggcagtgccggaggcgcagggacaacacgacggagctggagatggtgccggggagctccaccgcgttcggcacggcctactacggcctggtcgcggagcggagggcgctcttccactccgacgaggcgctgctcaggaacggggagaccagggcgctcgtctaccgctatagggacgcgccgtccgaggcggcgttcctcgcggaattcggggcgtccatgctcaacatgggcagggtgggcgtgctcaccggcgcccagggggagatcaggaagaggtgcgcctttgtcaactagctagcgatatgctggattgtactttgtaccctctcgccttaattaaaatttaaatgctggagtttcacctaaaaaaaa

SEQ ID NO: 33 - protein, 355 aa, Zea mays
MYTAMAARPLLLPPPVLLLLVVLAASSAAHGYGAYGYGDAAAELRVGFYKDSCPDAEAVVRRIVAKAVQEDPTANAPLLRLHFHDCFVRGCDGSVLVNSTRGNTAEKDAKPNHTLDAFDVIDDIKEALEKRCPGTVSCADILAIAARDAVSLATKVVTKGGWSRDGNLYQVETGRRDGRVSRAKEAVKNLPDSMDGIRKLIRRFASKNLSVKDLAVLSGAHAIGKSHCPSIAKRLRNFTAHRDSDPTLDGAYAAELRRQCRRRRDNTTELEMVPGSSTAFGTAYYGLVAERRALFHSDEALLRNGETRALVYRYRDAPSEAAFLAEFGASMLNMGRVGVLTGAQGEIRKRCAFVN

SEQ ID NO: 34 - DNA, 1391 bp, Zea mays
  CDS (103)...(1089)
ggacgagactcgcagctagctgacacggccgagaagcagcttgcattgcaggcgtagtacgtacccagcagcagctagcactagcagtccatcggagcgacgatggtgagaaggacggtgctggcggcgctgctggtggccgccgccctcgccggcggcgcgcgggcgcagctcaaggaggggttctacgactactcctgcccacaggcggagaagatcgtcaaggactacgtgaaggcgcacatcccccacgcgcccgacgtcgcctccaccctgctccgcacccacttccacgactgcttcgtcaggggctgcgacgcgtcagtgctgctcaacgcgacgggcggcagcgaggcggagaaggacgcggcgcccaacctgacgctgcgcggcttcggcttcatcgaccgcatcaaggcgctgctcgagaaggagtgccccggcgtggtgtcctgcgccgacatcgtcgcgctcgccgcccgcgactccgtcggcgtcatcggcggtccgttctggagcgtgccgacggggaggcgcgacggcaccgtgtccatcaagcaggaggcgctggaccagatccccgcgcccaccatgaacttcacccaactcctccagtccttccagaacaagagcctcaacctcgccgacctcgtctggctctcaggggctcacacgatcggcatctcccaatgcaactccttcagcgagcgcctgtacaacttcacggggcgcggcgggcccgacgacgcggacccgtcgctggacccgctgtacgccgcgaagttgcggctcaagtgcaagacgctgacggacaacacgacgatcgtggagatggaccccggcagcttccgcaccttcgacctgagctactaccgcggcgtgctcaagcggcggggcctgttccagtccgacgccgcgctcatcaccgacgccgcctccaaggccgacatcctcagcgtgatcaacgcgccgcccgaggtgttcttccaggtcttcgcgggctccatggtcaagatgggcgccatcgaggtcaagaccggctccgagggcgagatcaggaagcactgcgccctcgtcaacaagcactaggcggcggaattcatgcgggagatggctccattgctcgcaaaaaaatccttgtgagacacacaacatgctctgcatctgcaggcgttgtcgtcaccttggtggacgtagtacatcggtgcatggattatctgttgtttaatttgtacgttcagttcattctgtttcttgtattcttttggttcctttcctcttgtttattcatggatgatgaggtgtttctgttttattctctggttcagctgtaaccatgtaacatgtaaggtgcgcgttttgtcctaaaaaaaaaaaaaaaaaaaaaaaaa

SEQ ID NO: 35 - protein, 328 aa, Zea mays; identical to SEQ ID NO: 19

SEQ ID NO: 36 - DNA, 1476 bp, Zea mays
  CDS (259)...(1236)
accagctgcagccacaagcgcagcgctcgacagcctcaccagccgccatcactcgcggcagtacccggccgctgggactgcaagagtggggagtgagacgctgctgctgttgccgagcgcgaggagacactactactcactcaagagtcaagactcaacagcagcaggggcgaccgagctctgcgtgcgtggggagaaccggagaaggcagcagaggagggagggagggagcgcgtggaccaggctaggcgcagcagcatgggcgccgggatcagggtcctggcggcgctcttggcggcgctcgcggcagccaccgccggcctgacggcgcagctgcggcaggactactacgcggctgtgtgcccggacctggagagcatcgtgcgcgccgcggtgtccaagaaggtgcaggcgcagcccgtcgccgtgggcgccaccatccgcctcttcttccacgactgcttcgtcgagggctgcgacgcgtcggtgatcctggtgtccacggggaacaacacggcggagaaggaccacccgagcaacctctccctggccggcgacggcttcgacaccgtcatccaggccaaggcggccgtggacgccgtgccggcgtgcgccaaccaggtgtcgtgcgccgacatcctggcgctggccacccgggacgtcattgagctggctggcgggccgtcgtacgcggtggagctggggaggctggacgggctggtgtccatgtccaccaacgtcgacggcaagctgccgccgccgtccttcaacctggaccagctgacgagcattttcgccctcaacaacctgtcgcaggccgacatgattgctttatctgcggcgcacacggtggggttcgcgcactgcagcacgttctcggaccggatccagccgcagtcggtggacccgacgatgaacgcgacgtacgcggaggacctgcaggcggcgtgcccggcgggggtggaccccaacatcgcgctgcagctggaccccgtgacgccgcaggccttcgacaaccagtacttcgccaacctggtggacggccgggggctcttcgcctccgaccaggtgctcttctccgacgcgcggtcgcagcccaccgtggtggcgtgggcgcagaacgccaccgacttcgagcaggccttcgtcgacgccatcaccaggctcggccgcgtcggcgtcaagaccgacccgtcgctgggggacgtccgccgcgactgcgccttcctcaactgaaaaaggatgataagcgctagctagtaggtgtaagcatcggctggcactcacctggaggaagaggccggcttttggtcagtggtgacatctgatggatgtccaatcacgcaggatgccaaaaggcccacgcctacgcatatgaacggtgaaatatagcgaactctaaatagcaaacactgctggagggctcttggacgctaagaaacgctacatcttctcgcaaaaaaaaaaaaaaaaaaa

SEQ ID NO: 37 - protein, 325 aa, Zea mays
MGAGIRVLAALLAALAAATAGLTAQLRQDYYAAVCPDLESIVRAAVSKKVQAQPVAVGATIRLFFHDCFVEGCDASVILVSTGNNTAEKDHPSNLSLAGDGFDTVIQAKAAVDAVPACANQVSCADILALATRDVIELAGGPSYAVELGRLDGLVSMSTNVDGKLPPPSFNLDQLTSIFALNNLSQADMIALSAAHTVGFAHCSTFSDRIQPQSVDPTMNATYAEDLQAACPAGVDPNIALQLDPVTPQAFDNQYFANLVDGRGLFASDQVLFSDARSQPTVVAWAQNATDFEQAFVDAITRLGRVGVKTDPSLGDVRRDCAFLN